SEQUENCE LISTING (12 sequences)

SEQ ID NO: 1 - protein, 219 aa, Ustilago maydis
MLIFSVLMYLGLLLAGASALPNGLSPRNNAFCAGFGLSCKWECWCTAHGTGNELRYATAAGCGDHLSKSYYDARAGHCLFSDDLRNQFYSHCSSLNNNMSCRSLSKRTIQDSATDTVDLGAELHRDDPPPTASDIGKRGKRPRPVMCQCVDTTNGGVRLDAVTRAACSIDSFIDGYYTEKDGFCRAKYSWDLFTSGQFYQACLRYSHAGTNCQPDPQYE

SEQ ID NO: 2 - protein, 27 aa, Ustilago maydis
MLIFSVLMYLGLLLAGASALPNGLSPR

SEQ ID NO: 3 - protein, 27 aa, Artificial Sequence
  MsDef1 Export Signal Amino Acid Sequence
MEKKSLAGLCFLFLVLFVAQEIVVTEA

SEQ ID NO: 4 - DNA, 82 bp, Artificial Sequence
  MsDef1 Export Signal Amino Acid Sequence Nucleotide Coding Sequence
atggagaagaaatcactagctggcttatgcttcctcttcttggttctctttgttgcacaagaaattgtggtgacagaagcca

SEQ ID NO: 5 - protein, 192 aa, Artificial Sequence
  Wild-Type U. maydis Amino Acid Core KP6 Protoxin Sequence Without N-Terminal Sequence
NNAFCAGFGLSCKWECWCTAHGTGNELRYATAAGCGDHLSKSYYDARAGHCLFSDDLRNQFYSHCSSLNNNMSCRSLSKRTIQDSATDTVDLGAELHRDDPPPTASDIGKRGKRPRPVMCQCVDTTNGGVRLDAVTRAACSIDSFIDGYYTEKDGFCRAKYSWDLFTSGQFYQACLRYSHAGTNCQPDPQYE

SEQ ID NO: 6 - DNA, 576 bp, Artificial Sequence
  Wild-Type U. maydis Amino Acid Core KP6 Protoxin Sequence Without N-Terminal Sequence  Nucleotide Coding Sequence
aacaatgctttttgtgctggatttggtctctcttgcaagtgggaatgttggtgcacagcacacggaacgggcaatgaattacggtatgctaccgcagcaggatgcggagatcatctgtccaagtcttattacgatgctcgggccggccactgcctgttctctgacgaccttcgcaaccagttctacagccattgttcgtctctaaacaacaatatgtcgtgccggtcgttgtctaaacggactatccaagatagcgctaccgacacggtagacctcggtgccgagctccatagggatgacccgccccctactgctagtgacataggcaaacggggtaagaggcctagacctgttatgtgccaatgtgtagacacaacgaacggaggggttcgattagacgcggtgactagggcggcttgcagcatagactcgtttatcgacgggtactatacggaaaaggatgggttttgtagagctaaatattcctgggacttgtttacgagcggccagttctaccaggcatgtttgaggtactcacatgccgggaccaactgccaacctgacccgcagtatgaa

SEQ ID NO: 7 - protein, 219 aa, Artificial Sequence
  Chimeric U. maydis KP6 Protoxin Protein Sequence With N-Terminal MsDef1 Export Signal Amino Acid Sequence
MEKKSLAGLCFLFLVLFVAQEIVVTEANNAFCAGFGLSCKWECWCTAHGTGNELRYATAAGCGDHLSKSYYDARAGHCLFSDDLRNQFYSHCSSLNNNMSCRSLSKRTIQDSATDTVDLGAELHRDDPPPTASDIGKRGKRPRPVMCQCVDTTNGGVRLDAVTRAACSIDSFIDGYYTEKDGFCRAKYSWDLFTSGQFYQACLRYSHAGTNCQPDPQYE

SEQ ID NO: 8 - DNA, 657 bp, Artificial Sequence
  Chimeric U. maydis KP6 Protoxin Protein Sequence With N-Terminal MsDef1 Export Signal Amino Acid Sequence Nucleotide Coding Sequence
atggagaagaaatcactagctggcttatgcttcctcttcttggttctctttgttgcacaagaaattgtggtgacagaagccaacaatgctttttgtgctggatttggtctctcttgcaagtgggaatgttggtgcacagcacacggaacgggcaatgaattacggtatgctaccgcagcaggatgcggagatcatctgtccaagtcttattacgatgctcgggccggccactgcctgttctctgacgaccttcgcaaccagttctacagccattgttcgtctctaaacaacaatatgtcgtgccggtcgttgtctaaacggactatccaagatagcgctaccgacacggtagacctcggtgccgagctccatagggatgacccgccccctactgctagtgacataggcaaacggggtaagaggcctagacctgttatgtgccaatgtgtagacacaacgaacggaggggttcgattagacgcggtgactagggcggcttgcagcatagactcgtttatcgacgggtactatacggaaaaggatgggttttgtagagctaaatattcctgggacttgtttacgagcggccagttctaccaggcatgtttgaggtactcacatgccgggaccaactgccaacctgacccgcagtatgaa

SEQ ID NO: 9 - protein, 79 aa, Ustilago maydis
NNAFCAGFGLSCKWECWCTAHGTGNELRYATAAGCGDHLSKSYYDARAGHCLFSDDLRNQFYSHCSSLNNNMSCRSLSK

SEQ ID NO: 10 - DNA, 237 bp, Ustilago maydis
aacaatgctttttgtgctggatttggtctctcttgcaagtgggaatgttggtgcacagcacacggaacgggcaatgaattacggtatgctaccgcagcaggatgcggagatcatctgtccaagtcttattacgatgctcgggccggccactgcctgttctctgacgaccttcgcaaccagttctacagccattgttcgtctctaaacaacaatatgtcgtgccggtcgttgtctaaa

SEQ ID NO: 11 - protein, 81 aa, Ustilago maydis
GKRPRPVMCQCVDTTNGGVRLDAVTRAACSIDSFIDGYYTEKDGFCRAKYSWDLFTSGQFYQACLRYSHAGTNCQPDPQYE

SEQ ID NO: 12 - DNA, 247 bp, Ustilago maydis
ggtaagaggcctagacctgttatgtgccaatgtgtagacacaacgaacggaggggttcgattagacgcggtgactagggcggcttgcagcatagactcgtttatcgacgggtactatacggaaaaggatgggttttgtagagctaaatattcctgggacttgtttacgagcggccagttctaccaggcatgtttgaggtactcacatgccgggaccaactgccaacctgacccgcagtatgaataag

(19) United States
(12) Patent Application Publication
(10) Pub. No.: US 2015/0373995 A1
(43) Pub. Date: Dec. 31, 2015

(54) KP6 ANTIFUNGAL PROTEIN-INDUCED FUNGAL RESISTANCE IN FOOD CROPS

(71) Applicant: DONALD DANFORTH PLANT SCIENCE CENTER, ST. LOUIS, MO (US)

(72) Inventors: Thomas James Smith, St. Louis, MO (US); Dilip Maganial Shah, St. Louis, MO (US)

(21) Appl. No.: 14/850,455

(22) Filed: Sep. 10, 2015

Related U.S. Application Data
(63) Continuation of application No. PCT/US2014/023149, filed on Mar. 11, 2014.
(60) Provisional application No. 61/776,253, filed on Mar. 11, 2013.

(51) Int. Cl. A01N 63/04; C07K 14/375; A01N 37/46; C12N 15/82

(52) U.S. Cl. CPC A01N 63/04; C12N 15/8282; C12N 15/8227; C07K 14/375; A01N 37/46

(57) ABSTRACT

Provided are transgenic plants expressing KP6 antifungal protein and/or KP6 α and β polypeptides, exhibiting high levels of fungal resistance. Such transgenic plants contain a recombinant DNA construct comprising a heterologous signal peptide sequence operably linked to a nucleic acid sequence encoding these molecules. Also provided are methods of producing such plants, methods of protecting plants against fungal infection and damage, as well as compositions that can be applied to the locus of plants, comprising microorganisms expressing these molecules, or these molecules themselves, as well as pharmaceutical compositions containing these molecules. Human and veterinary therapeutic use of KP6 antifungal protein and/or KP6 α and β polypeptides are also encompassed by the invention.

### CROSS-REFERENCE TO RELATED APPLICATIONS

This application is a continuation of PCT Application No. PCT/US2014/023149 filed on Mar. 11, 2014, which claims the benefit of priority of U.S. Provisional Application Ser. No. 61/776,253, filed Mar. 11, 2013, the contents of which are herein incorporated by reference in their entirety.

### BACKGROUND OF THE INVENTION

1. Field of the Invention

The present invention relates to the use of Ustilago maydis KP6 antifungal protein to impart resistance to Fusarium sp. and other fungi in food crop plants, including methods for controlling pathogenic fungi employing this antifungal polypeptide. The antifungal polypeptide can be applied directly to a plant, applied to a plant in the form of micro-organisms that produce the polypeptide, or plants themselves can be genetically modified to produce the polypeptide. The present invention also relates to DNA sequences, microorganisms, plants, and compositions useful in these methods.

2. Description of Related Art

Protection of agriculturally important crops from pathogenic fungi is crucial in improving crop yields. Fungal infections are a particular problem in damp climates, and may become a major concern during crop storage, where such infections can result in spoilage and contamination of food or feed products with fungal toxins. Unfortunately, modern growing methods, harvesting, and storage systems can promote plant pathogen infections. In most cases, control of fungal infection using traditional breeding has met with limited success because natural resistance is most often organ-specific and involves numerous genes.

In addition, there are a number of pathogens that are soil-borne. This poses a particularly challenging threat since fungicides cannot penetrate into the soils to prevent infection in the root tissue, and once a field is contaminated, there is little that can be done to rectify the persistent infection. A case in point, Fusarium virguliforme (formally named Fusarium solani), is the major cause of soybean sudden death syndrome (SDS) in the United States. SDS is an economically important soybean disease that causes yield losses ranging from 5 to 15%. However, in individual fields, losses of up to 80% can be realized. In the United States between 2004 and 2006, an estimated 89.3 million bushels were lost to this disease, resulting in a net loss of approximately $587 million. SDS greatly reduces the seed size and pod number, resulting in lower seed weight and fewer seeds. Only a few soybean varieties derived from a narrow genetic base have some level of resistance, but no variety has shown immunity to this disease. SDS is a major concern to both farmers and seed suppliers, and is consistently placed in the top four causes for crop losses due to disease. Of even greater concern is that this disease continues to march northward into areas of the Midwest that were previously unaffected by SDS. To date, tillage and crop rotations have had limited and inconsistent impact on controlling the disease. Further, there is a synergistic effect of SDS and nematode infection, and nematodes may harbor the fungus over winter. Therefore, host resistance remains the most promising control strategy for SDS.

Ustilago maydis is a fungal pathogen of maize that causes corn smut. Some strains of U. maydis secrete killer toxins that are encoded by endogenous, noninfectious double-stranded RNA viruses in the cell cytoplasm, and which are capable of killing other susceptible strains of U. maydis. Only one form of the U. maydis killer toxin KP6 is known at this time. It has both a unique amino acid sequence as well as a wholly unique protein structure. The KP6 toxin contains two separate, non-covalently associated polypeptide chains, α (SEQ ID NO:9) and β (SEQ ID NO:11), having 79 and 81 amino acids, respectively, both of which are necessary for its killer activity, and which are separately secreted from U. maydis cells. The KP6 α and β polypeptides are processed from a 219 amino acid preprotoxin (SEQ ID NO:1) by a Kex 2p-like protease during export from the fungal cell. Signal peptidase cleaves after alanine 19 and Kex2p cleaves after amino acids 27, 107, and 138. The C-terminal arginine is removed from KP6 α, leaving a KP6 α of 79 amino acids and a KP6 β of 81 amino acids (J. Bruenn (2005), “The Ustilago maydis Killer Toxins”, Topics in Current Genetics, Vol. 11, M. J. Schmitt and R. Schaffrath (Eds.), Microbial Protein Toxins, pp. 157-174, Springer-Verlag, Berlin, Heidelberg, published online Jul. 22, 2004, DOI 10.1007/b100197).

As demonstrated by Peery et al. (1987) Mol. Cell Biol. 7:470-477, the α and β polypeptides can be synthesized separately. Thus, systemic production of functional KP6 antifungal toxin for plant protection requires that the host plant be able to correctly process the preprotoxin. The unprocessed preprotoxin may itself exhibit antifungal activity.

Koltin et al. (1975), “Specificity of Ustilago maydis Killer Proteins”, Appl. Environ. Microbiol. 30(4):694-696 discloses (Table 1) the sensitivity to Ustilago KP6 of the grass smuts Sorosporium consanguineum and a number of Ustilago species. Other Ustilago species, as well as Endothia parasitica, a number of Helminthosporium species, Saccharomyces cerevisiae, and Schizophyllum commune, were insensitive. The activity of KP6 against Fusarium species was not tested.

Kinal et al. (1995), “Processing and Secretion of a Virally Encoded Antifungal Toxin in Transgenic Tobacco Plants: Evidence for a Kex2p Pathway in Plants”, Plant Cell 7:677-688 discloses successful processing and secretion of KP6 antifungal toxin in transgenic tobacco plants containing the viral toxin cDNA under the control of a cauliflower mosaic virus promoter to produce functional KP6 toxin. While the authors suggest that systemic production of this viral killer toxin in crop plants may provide a new method of engineering biological control of fungal pathogens in crop plants, especially against a wide range of Ustilago species known as crop pathogens of maize, wheat, oats and barley, they do not disclose or suggest that the KP6 antifungal toxin has, or would have, any activity against Fusarium species. They note that U. maydis virus toxins have no toxic effects on cell types other than those of the Ustilaginales. The authors could not determine whether their transgenic tobacco plants were resistant to KP6-sensitive strains of U. maydis because tobacco is not normally susceptible to U. maydis infection, i.e., they did not demonstrate efficacy in planta against any fungal pathogen.

J. Bruenn (2005), “The Ustilago maydis Killer Toxins”, Topics in Current Genetics, Vol. 11, M. J. Schmitt and R. Schaffrath (Eds.), Microbial Protein Toxins, pp. 157-174, Springer-Verlag, Berlin, Heidelberg, published online Jul. 22, 2004, DOI 10.1007/b100197, discloses that KP6 has been expressed from cDNA clones in Ustilago, yeast, and plants (tobacco), and that the ability to express KP6 in the latter is surprising because the processing of the toxin varies in each system, and because a gene for Kex 2p has not been demonstrated in plant systems. The author notes that processing of KP6 in tobacco is much less efficient that that in Ustilago, since despite an abundance of mRNA, the plants produce very little toxin. No mention is made of the activity of KP6 against Fusarium.

There remains a need for simple, improved methods for the control of fungal infections in plants. The production of transgenic plants expressing KP6 as described herein provides such a simple, improved method by providing a novel and effective approach for controlling such pathogens, especially Fusarium species, in crop and other plants, while minimizing broad toxic effects against other cell types. This is a significant development since this is the first description of the activity of KP6 toxin against Fusarium species, and since current breeding programs have failed to produce resistant lines because fungal resistance generally requires multiple endogenous genes. The present method facilitates single gene resistance that is easily maintained in a variety of crops. The present method is also surprising since it was not known whether proteases in a variety of different plants are capable of correctly processing KP6 preprotoxin, and whether the KP6 α and β polypeptides would correctly associate to form a functional inhibitor in a variety of plants. Additional factors successfully demonstrated herein in using this technology in a broad range of plants include whether host plants would produce adequate mRNA transcript levels and protein levels to impart resistance; possible differences in post-translational processing, such as glycosylation, in different plant hosts that might adversely affect activity, fungal strain specificity, etc.; differences in KP6 secretion in different plants; undesirable proteolysis in different plants; possible inhibition by various intracellular components in different plants; and whether expression of KP6 would have any detrimental effects on host plant growth and/or development.

The inventors have previously demonstrated that KP4 toxin is highly efficacious in protecting maize against Ustilago maydis infection (Allen et al. (2011) Plant Biotech Journal 9:857-864). KP6 is completely different from KP4 in several respects. KP4 is a cytostatic protein in that its inhibition can be reversed upon washing the protein from the fungal cells (Gage et al. (2001) Molecular Microbiology 41:775-785; Gage et al. (2002) Molecular Pharmacology 61:936-944). Further, KP4 acts via calcium channels both directly (⁴⁵Ca²⁺ uptake experiments) and via electrophysiological experiments (Gu et al. (1995) Structure 3:805-814; Gage et al. (2001) and (2002), supra). In spite of this transient mode of action, KP4 is still efficacious at blocking fungal infections in maize. In contrast, the effects of KP6 are irreversible and independent of calcium (unpublished results). Thus, while KP4 provides good antifungal protection, KP6 is expected to provide a more permanent solution, including even clearing soils of persistent contamination via expression through the plant rhizome.

### SUMMARY OF THE INVENTION

The present inventors have discovered that KP6 antifungal protein, and KP6 α and β polypeptides, exhibit antifungal activity against Fusarium and other fungal species. Accordingly, the present invention provides:
1. A protein, comprising the amino acid sequence shown in SEQ ID NO:5 and a plant apoplast, vacuolar, or endoplasmic reticulum targeting amino acid sequence at its N-terminus. This protein can be an isolated, purified protein.
2. The protein of 1, wherein said targeting sequence comprises the amino acid sequence shown in SEQ ID NO:3.
3. A nucleotide sequence encoding said protein of 1 or 2.
4. The nucleotide sequence of 3, codon-optimized for expression in a plant of interest other than tobacco.
5. The nucleotide sequence of 3 or 4, wherein said plant other than tobacco is a food crop plant.
6. The nucleotide sequence of 5, wherein said food crop plant is selected from the group consisting of soybean, wheat, maize, and sugarcane.
7. A transgenic food crop plant, cells of which contain a protein comprising the amino acid sequence shown in SEQ ID NO:5.
8. The transgenic food crop plant of 7, wherein said protein further comprises a plant apoplast, vacuolar, or endoplasmic reticulum targeting amino acid sequence at its N-terminus.
9. The transgenic food crop plant of 7 or 8, wherein said protein is present in said cells in an antifungal effective amount.
10. The transgenic food crop plant of any one of 7-9, wherein said cells are root cells.
11. The transgenic food crop plant of any one of 7-10, wherein said protein inhibits damage to said plant caused by a species of Fusarium.
12. The transgenic food crop plant of 11, wherein said species of Fusarium is selected from the group consisting of Fusarium solani, Fusarium nivale, Fusarium oxysporum, Fusarium graminearum, Fusarium culmorum, Fusarium moniliforme, Fusarium roseum, Fusarium verticillioides, and Fusarium proliferatum.
13. The transgenic food crop plant of any one of 7-12, the genome of which further comprises:
   DNA encoding a plant defensin selected from the group consisting of MsDef1, MtDef2, MtDef4, Rs-AFP1, Rs-AFP2, and KP4, wherein said DNA is expressed and produces an anti-fungal effective amount of said defensin, and/or
   DNA encoding a Bacillus thuringiensis endotoxin, wherein said DNA is expressed and produces an anti-insect effective amount of said Bacillus thuringiensis endotoxin, and/or
   DNA encoding a protein that confers herbicide resistance to said food crop plant, wherein said DNA is expressed and produces an anti-herbicide effective amount of said protein that confers herbicide resistance.
14. The transgenic food crop plant of any one of 7-13, produced by a method comprising:
   a) inserting into the genome of a food crop plant cell a recombinant, double-stranded DNA molecule comprising, operably linked for expression:
      (i) a promoter sequence that functions in plant cells to cause the transcription of an adjacent coding sequence to RNA;
      (ii) a coding sequence encoding a protein comprising the amino acid sequence shown in SEQ ID NO:5 and a plant apoplast, vacuolar, or endoplasmic reticulum targeting amino acid sequence at its N-terminus;
      (iii) a 3′ non-translated sequence that functions in plant cells to cause transcriptional termination and the addition of polyadenylate nucleotides to the 3′ end of said transcribed RNA;
   b) obtaining a transformed food crop plant cell; and
   c) regenerating from said transformed food crop plant cell a genetically transformed food crop plant, cells of which express said protein.
15. The transgenic food crop plant of 14, wherein said protein is expressed in an antifungal effective amount in cells of said transformed food crop plant.
16. The transgenic food crop plant of 14 or 15, wherein said coding sequence comprises the nucleotide sequence shown in SEQ ID NO:8, or a codon-optimized version of SEQ ID NO:8 to optimize expression thereof in said plant.
17. The transgenic food crop plant of any one of 14-16, wherein said promoter is a root-specific promoter.
18. The transgenic food crop plant of 17, wherein said root-specific promoter is selected from the group consisting of RB7, RD2, ROOT1, ROOT2, ROOT3, ROOT4, ROOT5, ROOT6, ROOT7, and ROOT8.
19. The transgenic food crop plant of any one of 7-18, which is selected from the group consisting of maize, soybean, wheat, and sugarcane.
20. A part of said transgenic food crop plant of any one of 7-19.
21. The part of 20, which is selected from the group consisting of a protoplast, a cell, a tissue, an organ, a cutting, and an explant.
22. The part of 21, which is selected from the group consisting of an inflorescence, a flower, a sepal, a petal, a pistil, a stigma, a style, an ovary, an ovule, an embryo, a receptacle, a seed, a fruit, a stamen, a filament, an anther, a male or female gametophyte, a pollen grain, a meristem, a terminal bud, an axillary bud, a leaf, a stem, a root, a tuberous root, a rhizome, a tuber, a stolon, a corm, a bulb, an offset, a cell of said plant in culture, a tissue of said plant in culture, an organ of said plant in culture, and a callus.
23. Progeny of said transgenic food crop plant of any one of 7-19.
24. Seed of said transgenic food crop plant of any one of 7-19.
25. A transgenic food crop plant, cells of which comprise a nucleotide coding sequence encoding a protein comprising the amino acid sequence shown in SEQ ID NO:5.
26. The transgenic food crop plant of 25, wherein said nucleotide coding sequence further encodes a plant apoplast, vacuolar, or endoplasmic reticulum targeting amino acid sequence at the N-terminus of said protein.
27. The transgenic food crop plant of 25 or 26, wherein said protein is expressed in an antifungal effective amount in said cells.
28. The transgenic food crop plant of any one of 25-27, wherein said cells are root cells.
29. The transgenic food crop plant of any one of 25-28, produced by a method comprising:
   a) inserting into the genome of a food crop plant cell a recombinant, double-stranded DNA molecule comprising, operably linked for expression:
      (i) a promoter that functions in plant cells to cause transcription of an adjacent coding sequence to RNA;
      (ii) a coding sequence encoding a protein comprising the amino acid sequence shown in SEQ ID NO:5 and a plant apoplast, vacuolar, or endoplasmic reticulum targeting amino acid sequence at its N-terminus; and
      (iii) a 3′ non-translated region that functions in plant cells to cause transcriptional termination and the addition of polyadenylate nucleotides to the 3′ end of said transcribed RNA;
   b) obtaining a transformed food crop plant cell; and
   c) regenerating from said transformed food crop plant cell a genetically transformed food crop plant, cells of which express said protein.
30. The transgenic food crop plant of 29, wherein said protein is expressed in an antifungal effective amount in cells of said plant.
31. The transgenic food crop plant of 29 or 30, wherein said coding sequence comprises the nucleotide sequence shown in SEQ ID NO:8, or a codon-optimized version of SEQ ID NO:6 to optimize expression thereof in said plant.
32. The transgenic food crop plant of any one of 29-31, wherein said promoter is a root-specific promoter.
33. The transgenic food crop plant of 32, wherein said root-specific promoter is selected from the group consisting of RB7, RD2, ROOT1, ROOT2, ROOT3, ROOT4, ROOT5, ROOT6, ROOT7, and ROOT8.
34. The transgenic food crop plant of any one of 29-33, the genome of which further comprises:
   DNA encoding a plant defensin selected from the group consisting of MsDef1, MtDef2, MtDef4, Rs-AFP1, Rs-AFP2, and KP4, wherein said DNA is expressed and produces an anti-fungal effective amount of said defensin, and/or
   DNA encoding a Bacillus thuringiensis endotoxin, wherein said DNA is expressed and produces an anti-insect effective amount of said Bacillus thuringiensis endotoxin, and/or
   DNA encoding a protein that confers herbicide resistance to said food crop plant, wherein said DNA is expressed and produces an anti-herbicide effective amount of said protein that confers herbicide resistance.
35. The transgenic food crop plant of any one of 29-34, which is selected from the group consisting of maize, soybean, wheat, and sugarcane.
36. A food crop plant normally susceptible to damage from a species of Fusarium, cells of which contain a protein comprising the amino acid sequence shown in SEQ ID NO:5.
37. The food crop plant of 36, wherein said protein further comprises a plant apoplast, vacuolar, or endoplasmic reticulum targeting amino acid sequence at its N-terminus.
38. The food crop plant of 36 or 37, wherein said protein is present in said cells in an antifungal effective amount.
39. The food crop plant of any one of 36-38, wherein said cells are root cells.
40. The food crop plant of any one of 36-39, which is selected from the group consisting of maize, soybean, wheat, and sugarcane.
41. A food crop plant normally susceptible to damage from a species of Fusarium, cells of which contain α and β polypeptides comprising the amino acid sequences shown in SEQ ID NO:9 and SEQ ID NO:11, respectively.
42. The food crop plant of 41, wherein said α and β polypeptides are present together in an antifungal effective amount.
43. The food crop plant of 41 or 42, wherein said α and β polypeptides are present in stoichiometric proportion to one another.
44. The food crop plant of any one of 41-43, wherein said cells are root cells.
45. The food crop plant of any one of 41-44, which is resistant to damage by infection with a species of Fusarium.
46. The food crop plant of 45, wherein said species of Fusarium is selected from the group consisting of Fusarium solani, Fusarium nivale, Fusarium oxysporum, Fusarium graminearum, Fusarium culmorum, Fusarium moniliforme, Fusarium roseum, Fusarium verticillioides, and Fusarium proliferatum.
47. The food crop plant of any one of 41-46, further comprising DNA encoding:
   a plant defensin selected from the group consisting of MsDef1, MtDef2, MtDef4, Rs-AFP1, Rs-AFP2, and KP4, wherein said DNA is expressed and produces an anti-fungal effective amount of said defensin, and/or
   a Bacillus thuringiensis endotoxin, wherein said DNA is expressed and produces an anti-insect effective amount of said Bacillus thuringiensis endotoxin, and/or
   a protein that confers herbicide resistance to said food crop plant, wherein said DNA is expressed and produces an anti-herbicide effective amount of said protein that confers herbicide resistance.
48. The food crop plant of any one of 41-47, wherein said α and β polypeptides are present in apoplasts, vacuoles, or the endoplasmic reticulum of said cells of said food crop plant.
49. The food crop plant of any one of 41-48, which is selected from the group consisting of maize, soybean, wheat, and sugarcane.
50. The food crop plant of any one of 41-49, produced by a method comprising:
   a) inserting into the genome of a food crop plant cell a recombinant, double stranded DNA molecule comprising, operably linked for expression:
      (i) a promoter that functions in plant cells to cause transcription of an adjacent coding sequence to RNA;
      (ii) a coding sequence encoding a protein comprising the amino acid sequence shown in SEQ ID NO:5 and a plant apoplast, vacuolar, or endoplasmic reticulum targeting amino acid sequence at its N-terminus;
      (iii) a 3′ nontranslated region that functions in plant cells to cause transcriptional termination and the addition of polyadenylate nucleotides to the 3′ end of said transcribed RNA;
   b) obtaining a transformed food crop plant cell; and
   c) regenerating from said transformed food crop plant cell a genetically transformed food crop plant, cells of which express said protein.
51. The food crop plant of 50, wherein said protein is expressed in an antifungal effective amount in cells of said plant.
52. The food crop plant of 50 or 51, wherein said coding sequence encoding said protein comprises the nucleotide sequence shown in SEQ ID NO:8, or a codon-optimized version of SEQ ID NO:8 to optimize expression thereof in said plant.
53. The food crop plant of any one of 50-52, wherein said promoter is a root-specific promoter.
54. The food crop plant of 53, wherein said root-specific promoter is selected from the group consisting of RB7, RD2, ROOT1, ROOT2, ROOT3, ROOT4, ROOT5, ROOT6, ROOT7, and ROOT8.
55. The food crop plant of any one of 50-54, further comprising DNA encoding:
   a plant defensin selected from the group consisting of MsDef1, MtDef2, MtDef4, Rs-AFP1, Rs-AFP2, and KP4, wherein said DNA is expressed and produces an anti-fungal effective amount of said defensin, and/or
   a Bacillus thuringiensis endotoxin, wherein said DNA is expressed and produces an anti-insect effective amount of said Bacillus thuringiensis endotoxin, and/or
   a protein that confers herbicide resistance to said food crop plant, wherein said DNA is expressed and produces an anti-herbicide effective amount of said protein that confers herbicide resistance.
56. A method of preventing, treating, controlling, reducing, or inhibiting Fusarium damage to a Fusarium-susceptible food crop plant, comprising providing to the locus of said Fusarium-susceptible food crop plant an antifungal effective amount of a combination of polypeptides α (SEQ ID NO:9) and β (SEQ ID NO:11).
57. The method of 56, wherein said α and β polypeptides are provided to said Fusarium-susceptible food crop plant locus by expressing DNA encoding said polypeptides within cells of said Fusarium-susceptible food crop plant.
58. The method of 57, wherein said DNA encoding said α and β polypeptides comprises a nucleotide sequence encoding a protein comprising the amino acid sequence shown in SEQ ID NO:5.
59. The method of 58, wherein said DNA further comprises a nucleotide sequence encoding a plant apoplast, vacuolar, or endoplasmic reticulum targeting amino acid sequence at the N-terminus of SEQ ID NO:5.
60. The method of any one of 56-59, wherein said cells are root cells.
61. The method of 56, wherein said α and β polypeptides are provided to said Fusarium-susceptible food crop plant locus by plant colonizing microorganisms that produce said polypeptides.
62. The method of 56, wherein said α and β polypeptides are provided to said Fusarium-susceptible food crop plant locus by applying a composition comprising plant colonizing microorganisms that produce said polypeptides, or by applying said polypeptides themselves thereto.
63. The method of 62, wherein said composition comprising said polypeptides themselves comprises said α and β polypeptides in stoichiometric proportion to one another.
64. The method of 62 or 63, wherein each of said compositions comprises an agriculturally acceptable diluent, excipient, or carrier.
65. The method of any one of 56-64, wherein said Fusarium is a species selected from the group consisting of Fusarium solani, Fusarium nivale, Fusarium oxysporum, Fusarium graminearum, Fusarium culmorum, Fusarium moniliforme, Fusarium roseum, Fusarium verticillioides, and Fusarium proliferatum.
66. The method of any one of 56-65, wherein said Fusarium-susceptible food crop plant is selected from the group consisting of maize, soybean, wheat, and sugarcane.
67. A method of preventing, treating, controlling, or reducing, inhibiting Fusarium damage to a Fusarium-susceptible food crop plant, comprising expressing DNA comprising a nucleotide sequence encoding a protein comprising the amino acid sequence shown in SEQ ID NO:5 in cells thereof at a level sufficient to inhibit damage to said Fusarium-susceptible food crop plant caused by a species of Fusarium.
68. The method of 67, wherein said protein is targeted to apoplasts, vacuoles, or the endoplasmic reticulum of cells of said Fusarium-susceptible food crop plant.
69. The method of 67 or 68, wherein said protein is encoded by a nucleotide sequence comprising the nucleotide sequence shown in SEQ ID NO:8, or a codon-optimized version of SEQ ID NO:8 to optimize expression thereof in said plant.
70. The method of any one of 67-69, wherein said cells are root cells.
71. The method of any one of 67-70, wherein said Fusarium is a species selected from the group consisting of Fusarium solani, Fusarium nivale, Fusarium oxysporum, Fusarium graminearum, Fusarium culmorum, Fusarium moniliforme, Fusarium roseum, Fusarium verticillioides, and Fusarium proliferatum.
72. The method of any one of 67-71, wherein said Fusarium-susceptible food crop plant is selected from the group consisting of maize, soybean, wheat, and sugarcane.
73. A method of inhibiting Fusarium damage to a Fusarium-susceptible food crop plant, comprising:
   a) inserting into the genome of a food crop plant cell a recombinant, double stranded DNA molecule comprising, operably linked for expression:
      (i) a promoter that functions in plant cells to cause the transcription of an adjacent coding sequence to RNA;
      (ii) a coding sequence comprising a nucleotide sequence encoding a protein comprising the amino acid sequence shown in SEQ ID NO:5; and
      (iii) a 3′ nontranslated region that functions in said plant cells to cause transcriptional termination and the addition of polyadenylate nucleotides to the 3′ end of said transcribed RNA;
   b) obtaining a transformed food crop plant cell; and
   c) regenerating from said transformed food crop plant cell a genetically transformed food crop plant, cells of which express said protein.
74. The method of 73, wherein said protein is expressed in an antifungal amount in cells of said transformed food crop plant.
75. The method of 73 or 74, wherein said protein is targeted to apoplasts, vacuoles, or the endoplasmic reticulum of cells of said food crop plant.
76. The method of any one of 73-75, wherein said nucleotide sequence encoding said protein comprises the nucleotide sequence shown in SEQ ID NO:8, or a codon-optimized version of SEQ ID NO:8 to optimize expression thereof in said plant.
77. The method of any one of 73-76, wherein said promoter is a root-specific promoter.
78. The method of 77, wherein said root-specific promoter is selected from the group consisting of RB7, RD2, ROOT1, ROOT2, ROOT3, ROOT4, ROOT5, ROOT6, ROOT7, and ROOT8.
79. The method of any one of 73-78, wherein said Fusarium is a species selected from the group consisting of Fusarium solani, Fusarium nivale, Fusarium oxysporum, Fusarium graminearum, Fusarium culmorum, Fusarium moniliforme, Fusarium roseum, Fusarium verticillioides, and Fusarium proliferatum.
80. The method of any one of 73-79, wherein said Fusarium-susceptible food crop plant is selected from the group consisting of maize, soybean, wheat, and sugarcane.
81. A method of combating, preventing, treating, controlling, reducing, or inhibiting a species of Fusarium, comprising contacting said Fusarium species with a composition comprising an antifungal effective amount of a combination α and β polypeptides comprising the amino acid sequences shown in SEQ ID NO:9 and SEQ ID NO:11, respectively.
82. The method of 81, wherein said α and β polypeptides are present in said composition in stoichiometric proportion to one another.
83. The method of 81 or 82, wherein said composition comprises said combination of α and β polypeptides, and an agriculturally acceptable carrier, diluent, or excipient.
84. The method of 81, wherein said composition comprises microorganisms expressing said α and β polypeptides.
85. A method of combating, preventing, treating, controlling, reducing, or inhibiting fungal damage to a food crop plant, comprising:
   a) inserting into the genome of a food crop plant cell a recombinant, double-stranded DNA molecule comprising, operably linked for expression:
      (i) a promoter sequence that functions in plant cells to cause the transcription of an adjacent coding sequence to RNA;
      (ii) a coding sequence that encodes a protein comprising the amino acid sequence shown in SEQ ID NO:5; and
      (iii) a 3′ non-translated sequence that functions in plant cells to cause transcriptional termination and the addition of polyadenylation nucleotides to the 3′ end of said transcribed RNA;
   b) obtaining a transformed food crop plant cell; and
   c) regenerating from said transformed food crop plant cell a genetically transformed food crop plant, cells of which express said protein.
86. The method of 85, wherein said protein is expressed in an antifungal effective amount in cells of said transformed food crop plant.
87. The method of 85 or 86, wherein said coding sequence comprises a nucleotide sequence encoding a protein comprising the amino acid sequence shown in SEQ ID NO:5 and a plant apoplast, vacuolar, or endoplasmic reticulum targeting amino acid sequence at its N-terminus.
88. The method of 87, wherein said protein coding sequence comprises the nucleotide sequence shown in SEQ ID NO:8, or a codon-optimized version of SEQ ID NO:8 to optimize expression thereof in said plant.
89. The method of any one of 85-88, wherein said promoter is a root-specific promoter.
90. The method of 89, wherein said root-specific promoter is selected from the group consisting of RB7, RD2, ROOT1, ROOT2, ROOT3, ROOT4, ROOT5, ROOT6, ROOT7, and ROOT8.
91. The method of any one of 85-90, wherein said Fusarium is a species selected from the group consisting of Fusarium solani, Fusarium nivale, Fusarium oxysporum, Fusarium graminearum, Fusarium culmorum, Fusarium moniliforme, Fusarium roseum, Fusarium verticillioides, and Fusarium proliferatum.
92. The method of any one of 85-91, wherein said Fusarium-susceptible food crop plant is selected from the group consisting of maize, soybean, wheat, and sugarcane.
93. A method of combating, preventing, treating, controlling, reducing, or inhibiting damage to a food crop plant caused by a fungus, comprising:
   transforming a food crop plant with a DNA molecule encoding a protein comprising the amino acid sequence shown in SEQ ID NO:5 and a plant apoplast, vacuolar, or endoplasmic reticulum targeting amino acid sequence at its N-terminus to produce a transformed food crop plant,
   wherein cells of said transformed food crop plant produce said protein, and
   wherein said transformed food crop plant exhibits reduced fungal damage as compared to the fungal damage of an otherwise identical, untransformed control food crop plant that does not produce said protein when both plants are contacted with similar amounts of said fungus and are grown under the same conditions.
94. The method of 93, wherein said cells are root cells.
95. The method of 93 or 94, wherein said fungus is a species of Fusarium.
96. The method of 95, wherein said species of Fusarium is selected from the group consisting of Fusarium solani, Fusarium nivale, Fusarium oxysporum, Fusarium graminearum, Fusarium culmorum, Fusarium moniliforme, Fusarium roseum, Fusarium verticillioides, and Fusarium proliferatum.
97. The method of any one of 93-96, wherein said food crop plant is selected from the group consisting of maize, soybean, wheat, and sugarcane.
98. A method of reducing or inhibiting Fusarium contamination of soil, comprising cultivating in said soil transgenic plants expressing a protein comprising the amino acid sequence shown in SEQ ID NO:5 in cells of roots of said transgenic plants.
99. The method of 98, wherein said root cells produce said protein in an anti-Fusarium effective amount.
100. The method of 98 or 99, wherein said protein is targeted to apoplasts, vacuoles, or the endoplasmic reticulum of said root cells.
101. The method of any one of 98-100, wherein said Fusarium is a species selected from the group consisting of Fusarium solani, Fusarium nivale, Fusarium oxysporum, Fusarium graminearum, Fusarium culmorum, Fusarium moniliforme, Fusarium roseum, Fusarium verticillioides, and Fusarium proliferatum.
102. The method of any one of 98-101, wherein said transgenic plants are transgenic food crop plants.
103. The method of 102, wherein said transgenic food crop plants are selected from the group consisting of maize, soybean, wheat, and sugarcane.
104. A recombinant, double-stranded DNA molecule comprising, operatively linked for expression:
   a) a promoter that functions in plant cells to cause transcription of an adjacent coding sequence to RNA;
   b) a nucleotide sequence encoding a protein comprising the amino acid sequence shown in SEQ ID NO:5 and a plant apoplast, vacuolar, or endoplasmic reticulum targeting amino acid sequence at its N-terminus; and
   c) a 3′ non-translated sequence that functions in plant cells to cause transcriptional termination and the addition of polyadenylate nucleotides to the 3′ end of said transcribed RNA.
105. The recombinant, double-stranded DNA molecule of 104, wherein said promoter is a root-specific promoter.
106. The recombinant, double-stranded DNA molecule of 105, wherein said root-specific promoter is selected from the group consisting of RB7, RD2, ROOT1, ROOT2, ROOT3, ROOT4, ROOT5, ROOT6, ROOT7, and ROOT8.
107. The recombinant, double-stranded DNA molecule of any one of 104-106, which is codon-optimized for expression in a plant of interest.
108. The recombinant, double-stranded DNA molecule of 107, wherein said plant of interest is selected from the group consisting of maize, soybean, wheat, and sugarcane.
109. An expression construct, comprising a recombinant, double-stranded DNA molecule comprising, operably linked for expression:
   a) a promoter that functions in plant cells to cause the transcription of an adjacent coding sequence to RNA;
   b) a nucleotide sequence encoding a protein comprising the amino acid sequence shown in SEQ ID NO:5 and a plant apoplast, vacuolar, or endoplasmic reticulum targeting amino acid sequence at its N-terminus; and
   c) a 3′ nontranslated region that functions in plant cells to cause transcriptional termination and the addition of polyadenylate nucleotides to the 3′ end of said transcribed RNA.
110. The expression construct of 109, wherein said promoter is a root-specific promoter.
111. The expression construct of 110, wherein said root-specific promoter is selected from the group consisting of RB7, RD2, ROOT1, ROOT2, ROOT3, ROOT4, ROOT5, ROOT6, ROOT7, and ROOT8.
112. The expression construct of any one of 109-111, wherein said recombinant, double-stranded DNA molecule is codon-optimized for expression in a plant of interest.
113. The expression construct of 112, wherein said plant of interest is selected from the group consisting of maize, soybean, wheat, and sugarcane.
114. A plant transformation vector, comprising said recombinant, double-stranded DNA molecule of any one of 104-108, or the expression construct of any one of 109-113, and a selectable marker for selection of transformed plant cells.
115. An antifungal composition, comprising a combination of α and β polypeptides comprising the amino acid sequences shown in SEQ ID NOs:9 and 11, respectively.
116. The antifungal composition of 115, wherein said α and β polypeptides are present together in an antifungal effective amount.
117. The antifungal composition of 115 or 116, wherein said α and β polypeptides are present in stoichiometric proportion to one another.
118. The antifungal composition of any one of 115-117, further comprising an agriculturally or pharmaceutically acceptable carrier, diluent, or excipient.
119. The antifungal composition of any one of 115-118, wherein said α and β polypeptides are present together in a concentration in the range of from about 0.1 microgram per milliliter to about 500 milligrams per milliliter.
120. The antifungal composition of any one of 115-118, wherein said α and β polypeptides are present together in a concentration in the range of from about 5 micrograms per milliliter to about 250 milligrams per milliliter.
121. The antifungal composition of any one of 115-120, having a pH in the range of from about 3 to about 9.
122. The antifungal composition of any one of 115-121, formulated with one or more additives selected from the group consisting of an inert material, a surfactant, and a solvent.
123. The antifungal composition of any one of 115-122, formulated in a mixture of one or more other active agents selected from the group consisting of a pesticidally active substance, a fertilizer, an insecticide, an attractant, a sterilizing agent, an acaricide, a nematocide, a herbicide, and a growth regulator.
124. The antifungal composition of 123, wherein said pesticidally active substance is selected from the group consisting of a fungal antibiotic and a chemical fungicide.
125. The antifungal composition of 124, wherein said fungal antibiotic or chemical fungicide is selected from the group consisting of a polyoxine, a nikkomycine, a carboxyamide, an aromatic carbohydrate, a carboxine, a morpholine, a sterol biosynthesis inhibitor, and an organophosphate.
126. Use of said antifungal composition of any one of 115-125 to inhibit the growth of a fungal species.
127. The use of 126, wherein said fungal species is a Fusarium species.
128. The use of 127, wherein said Fusarium species is selected from the group consisting of Fusarium solani, Fusarium nivale, Fusarium oxysporum, Fusarium graminearum, Fusarium culmorum, Fusarium moniliforme, Fusarium roseum, Fusarium verticillioides, and Fusarium proliferatum.
129. The antifungal composition of any one of 115-125 for use in inhibiting the growth of a fungal species.
130. The use of 129, wherein said fungal species is a Fusarium species.
131. The use of 130, wherein said Fusarium species is selected from the group consisting of Fusarium solani, Fusarium nivale, Fusarium oxysporum, Fusarium graminearum, Fusarium culmorum, Fusarium moniliforme, Fusarium roseum, Fusarium verticillioides, and Fusarium proliferatum.
132. The antifungal composition of any one of 115-117, provided to a plant locus by colonizing microorganisms producing said α and β polypeptides.
133. The antifungal composition of any one of 115-117, wherein said α and β polypeptides are expressed from DNA encoding said polypeptides within cells of a transgenic food crop plant.
134. A method of controlling or inhibiting Fusarium, comprising contacting said Fusarium with a transgenic food crop plant, cells of which comprise and express a nucleotide sequence encoding a protein comprising the amino acid sequence shown in SEQ ID NO:5 and a plant apoplast, vacuolar, or endoplasmic reticulum targeting amino acid sequence at its N-terminus.
135. The method of 134, wherein said protein is expressed in cells of roots of said transgenic food crop plant.
136. The method of 135, wherein said protein is expressed by said root cells in an antifungal effective amount.
137. The method of any one of 134-136, wherein said Fusarium is a species selected from the group consisting of Fusarium solani, Fusarium nivale, Fusarium oxysporum, Fusarium graminearum, Fusarium culmorum, Fusarium moniliforme, Fusarium roseum, Fusarium verticillioides, and Fusarium proliferatum.
138. The method of any one of 134-137, wherein said transgenic food crop plant is selected from the group consisting of maize, soybean, wheat, and sugarcane.

Further scope of the applicability of the present invention will become apparent from the detailed description and drawing(s) provided below. However, it should be understood that the detailed description and specific examples, while indicating preferred embodiments of the invention, are given by way of illustration only since various changes and modifications within the spirit and scope of the invention will become apparent to those skilled in the art from this detailed description.

### BRIEF DESCRIPTION OF THE DRAWINGS

The above and other aspects, features, and advantages of the present invention will be better understood from the following detailed descriptions taken in conjunction with the accompanying drawing(s), all of which are given by way of illustration only, and are not limitative of the present invention, in which:

FIG. 1 shows transformation vector AKK/FMV/KP6 used to make KP6-expressing soybean lines. “LB”=T-DNA left border; “T-DNA RB”=T-DNA right border; “tNOS”=Nopaline synthase terminator sequence; “FMV”=Figwort mosaic virus 35S; “SU intron”=Super ubiquitin intron; “BAR”=Bialophos resistance gene; “NOS”=Nopaline synthase promoter; “Ori”=Origin of replication; “TraF”=Transfer F; “Tet(R)”=tetracycline resistance gene; “Kan(R)”=Kanamycin resistance gene; “TrfA”=T-DNA replication factor, and “KP6” represents the chimeric MsDef1/KP6 protein (SEQ ID NO:8).

FIG. 2 shows the results of the experiment described in Example 4, and discloses that KP6 can be expressed in an active form in both wheat (left image) and in soybean (right image) as evidenced by the zones of inhibition surrounding each explant. These assays are performed by simply placing pieces of transgenic leaf material in the agar wells. The agar contains the P2 strain of Ustilago maydis that is sensitive to KP6. The ‘+’ mark on the left image denotes the application of purified KP6 protein, and the ‘WT’ on the right image denotes the placement of the original, non-transgenic ‘Jack’ variety of soybean. The numbers in each panel indicate different transformation events.

FIG. 3 shows root samples of adjacent plots as described in Example 8 assayed for F. virguliforme contamination. Portions of the roots from field-dried material were collected, rehydrated for several hours in distilled water, and placed onto agar plates containing high levels of antibiotics to kill everything but the fungus. The vector control (transgenic soybeans made using the transformation vector without the KP6 transgene), and null lines (lines that went through the transformation process but were found not to have the KP6 transgene by PCR analysis) exhibit a much greater level of contamination compared to that of the transgenic lines (KP6-23 and KP6-24) even though they were only inches apart in the field. In this figure, the small black pieces represent portions of the roots from the field trials. The white plaques are colonies of F. virguliforme.

### DETAILED DESCRIPTION OF THE INVENTION

The following detailed description of the invention is provided to aid those skilled in the art in practicing the present invention. Even so, the following detailed description should not be construed to unduly limit the present invention, as modifications and variations in the embodiments herein discussed may be made by those of ordinary skill in the art without departing from the spirit or scope of the present inventive discovery.

The contents of each of the references discussed in this specification, including the references cited therein, are herein incorporated by reference in their entirety.

Any feature, or combination of features, described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

The amino acid and nucleotide sequences of the KP6 protein and α and β polypeptides, as well as those of the other elements useful in the constructs, methods, and organisms of the present invention, can be found at the end of the specification. All the amino acid and nucleotide sequences encompassed by the present invention include sequences consisting of, consisting essentially of, or comprising those specifically disclosed. As would be appreciated by one of ordinary skill in the art, as a result of the degeneracy of the genetic code, there are many nucleotide sequences that encode the peptide and protein molecules disclosed herein. Some of these polynucleotides may bear minimal homology to the nucleotide sequence of any native coding sequence.

In addition, polynucleotides that vary due to differences in codon usage are specifically contemplated by the present invention, and include those that are optimized for expression in monocots, dicots, yeasts, or bacteria. Nakamura et al. (2000) Nucl. Acids Res. 28(1):292 discusses the incorporation of preferred codons to enhance the expression of polynucleotides in various organisms. Codon usage in various monocot or dicot genes has been disclosed in Kawabe and Miyashita (2003) “Patterns of codon usage bias in three dicot and four monocot plant species”, Genes Genet. Syst. 78:343-352, and in Murray et al. (1989) “Codon Usage in Plant Genes” NAR 17:477-498. Methods for optimizing codon usage in plants are also disclosed in U.S. Pat. Nos. 5,500,365; 5,689,052; 5,500,365; and 5,689,052.

The present invention provides transgenic plants, plant-colonizing microorganisms, and compositions that can be applied to plants, capable of inhibiting the growth and development of pathogenic fungi, thereby resisting infection and damage caused by such fungi. In some embodiments, transgenic plants can be produced by introducing a DNA construct of the invention into a plant, a plant cell, or plant tissue or organ, and obtaining a transgenic plant comprising the DNA construct that expresses a plant pathogenic fungus inhibitory, i.e., antifungal effective, amount of KP6 antifungal protein or KP6 α and β polypeptides. Preferred plants of the invention are food crop plants, defined below, including monocots and dicots. Transgenic monocots of the invention can be selected from the group consisting of barley, maize, corn, flax, oat, rice, rye, sorghum, turf grass, sugarcane, and wheat. Transgenic dicot plants of the invention can be selected from the group consisting of alfalfa, Arabidopsis, barrel medic, banana, broccoli, bean, cabbage, canola, carrot, cassava, cauliflower, celery, citrus, cotton, a cucurbit, eucalyptus, garlic, grape, onion, lettuce, pea, peanut, pepper, potato, poplar, pine, sunflower, safflower, soybean, strawberry, sugar beet, sweet potato, tobacco, and tomato.

KP6 antifungal protein, the α and β polypeptides disclosed herein, and biologically functional equivalents thereof, are expected to be useful in controlling a wide variety of different fungi on crop plants. These fungi include those in the following genera and species: Alternaria (Alternaria brassicola; Alternaria solani); Ascochyta (Ascochyta pisi); Botrytis (Botrytis cinerea); Cercospora (Cercospora kikuchii; Cercospora zeae-maydis); Colletotrichum (Colletotrichum lindemuthianum); Diplodia (Diplodia maydis); Erysiphe (Erysiphe graminis f. sp. graminis; Erysiphe graminis f. sp. hordei); Fusarium (Fusarium nivale; Fusarium oxysporum; Fusarium graminearum; Fusarium culmorum; Fusarium solani; Fusarium moniliforme; Fusarium verticillioides; Fusarium roseum; Fusarium proliferatum); Gaeumanomyces (Gaeumanomyces graminis f. sp. tritici); Helminthosporium (Helminthosporium turcicum; Helminthosporium carbonum; Helminthosporium maydis); Macrophomina (Macrophomina phaseolina; Maganaporthe grisea); Nectria (Nectria heamatococca); Peronospora (Peronospora manshurica; Peronospora tabacina); Phakopsora (Phakopsora pachyrhizi); Phoma (Phoma betae); Phymatotrichum (Phymatotrichum omnivorum); Phytophthora (Phytophthora cinnamomi; Phytophthora cactorum; Phytophthora phaseoli; Phytophthora parasitica; Phytophthora citrophthora; Phytophthora megasperma f. sp. sojae; Phytophthora infestans); Plasmopara (Plasmopara viticola); Podosphaera (Podosphaera leucotricha); Puccinia (Puccinia sorghi; Puccinia striiformis; Puccinia graminis f. sp. tritici; Puccinia asparagi; Puccinia recondite; Puccinia arachidis); Pythium (Pythium aphanidermatum); Pyrenophora (Pyrenophora tritici-repentens); Pyricularia (Pyricularia oryzae); Pythium (Pythium ultimum); Rhizoctonia (Rhizoctonia solani; Rhizoctonia cerealis); Scerotium (Scerotium rolfsii); Sclerotinia (Sclerotinia sclerotiorum); Septoria (Septoria lycopersici; Septoria glycines; Septoria nodorum; Septoria tritici); Thielaviopsis (Thielaviopsis basicola); Uncinula (Uncinula necator); Venturia (Venturia inaequalis); Verticillium (Verticillium dahliae; Verticillium alboatrum).

A plant pathogenic fungus inhibitory amount (antifungal effective amount) of KP6 polypeptide, or a combination of the α and β polypeptides, is at least about 0.05 PPM, at least about 0.5 PPM, at least about 1.0 PPM, or at least about 2.0 PPM, where PPM are “parts per million” of KP6 protein or the α and β polypeptides present in fresh weight plant tissue, where 1 microgram of KP6 protein, or a combination of α and β polypeptides, per 1 gram of fresh weight plant tissue represents a concentration of 1 PPM.

In transgenic food crop plants of the invention, the growth of a variety of different plant pathogenic fungi is inhibited Inhibition of damage by pathogenic fungi can also be achieved by applying transformed plant-colonizing microorganisms, for example Pseudomonas fluorescens (U.S. Pat. No. 5,229,112), to the locus of plants. Compositions comprising such transformed plant-colonizing microorganisms, and/or comprising KP6 antifungal protein itself or a combination of the α and β polypeptides, can also be applied to the locus of plants to achieve fungal inhibition. Plant pathogenic fungi inhibited by the present methods and compositions include, but are not limited to, an Alternaria sp., an Ascochyta sp., a Botrytis sp.; a Cercospora sp., a Colletotrichum sp., a Diplodia sp., an Erysiphe sp., a Fusarium sp., Gaeumanomyces sp., Helminthosporium sp., Macrophomina sp., a Nectria sp., a Peronospora sp., a Phakopsora sp., a Phoma sp., a Phymatotrichum sp., a Phytophthora sp., a Plasmopara sp., a Puccinia sp., a Podosphaera sp., a Pyrenophora sp., a Pyricularia sp, a Pythium sp., a Rhizoctonia sp., a Scerotium sp., a Sclerotinia sp., a Septoria sp., a Thielaviopsis sp., an Uncinula sp, a Venturia sp., and a Verticillium sp.

In some embodiments, the invention includes transgenic food crop plants comprising a recombinant nucleic acid construct, or constructs, comprising a promoter operably linked to a nucleic acid encoding a heterologous signal peptide that is operably linked to a non-native nucleic acid encoding a KP6 antifungal protein, or the α and β polypeptides, that is operably linked to a polyadenylation sequence, wherein the transgenic plant expresses KP6 protein, or the α and β polypeptides. Depending on the fungus to which protection is sought, these molecules can be expressed in any tissue or organ in the plant where the fungus attacks. In the case of Fusarium for example, a preferred site for expression is in the roots. In the case of those fungi that infect by entering external plant surfaces, accumulation of KP6 protein, or the α and β polypeptides, in the apoplast is preferred, and can provide for at least about 50% inhibition of a plant pathogenic fungal infection compared to that in an otherwise identical, non-transgenic, control plant that lacks the recombinant nucleic acid construct. In different embodiments, the transgenic plant provides for at least about 75%, at least about 80%, at least about 85%, at least about 90%, or at least about 95% inhibition of a plant pathogenic fungal infection compared to that in an otherwise identical, non-transgenic, control plant that lacks the recombinant nucleic acid construct. In certain embodiments, the transgenic plant provides for at least about 50% inhibition of a biotrophic plant pathogenic fungus and/or at least about 50% inhibition of a necrotrophic plant pathogenic fungus. In certain embodiments, the biotrophic plant pathogenic fungus that is inhibited by the transgenic plants is selected from the group consisting of Ustilago species, Podosphaera species, Erysiphe species, Phakopsora species, and Puccinia species. In certain embodiments, the necrotrophic plant pathogenic fungus that is inhibited by the transgenic plants is selected from the group consisting of Alternaria species, Botrytis species, Colletotrichum species, Cercospora species, Fusarium species, Phoma species, Phytophthora species, Pythium species, Sclerotinia species, and Verticillium species. In certain embodiments, the transgenic plant is a monocot or a dicot, and the non-native nucleic acid sequence comprises one or more non-native codons that are more abundant in monocot plant genes and/or one or more non-native codons that are more abundant in dicot plant genes to optimize expression and accumulation.

In any of the aforementioned embodiments, the transgenic food crop plants can further comprise additional recombinant nucleic acid constructs, usually DNA, that provide for expression of MsDef1; MtDef2; MtDef4; Rs-AFP1; Rs-AFP2; KP4; a Bacillus thuringiensis endotoxin, wherein the polypeptide- or protein-encoding DNA of the construct is expressed and produces an anti-insect effective amount of the Bacillus thuringiensis endotoxin, and/or polypeptide- or protein-encoding DNA encoding a protein that confers herbicide resistance to the food crop plant, wherein the DNA is expressed and produces an anti-herbicide effective amount of the polypeptide or protein that confers herbicide resistance.

Simultaneous co-expression of multiple antifungal and/or other anti-pathogen proteins in plants is advantageous in that it exploits more than one mode of control of plant pathogens. This may, where two or more antifungal proteins are expressed, minimize the possibility of developing resistant fungal species, broaden the scope of resistance, and potentially result in a synergistic antifungal effect, thereby enhancing the level of resistance. Simultaneous co-expression of KP6 and/or the α and β polypeptides, with KP4 antifungal protein (disclosed in WO 2012/012480), may be especially useful in this regard and is specifically contemplated herein.

In any of the aforementioned embodiments, the heterologous signal peptide can be a signal peptide of a plant gene, which can be a dicot or monocot plant gene.

In other embodiments, the signal peptide can be from a defensin gene, for example from the plant defensin MsDef1. The signal peptide can have an amino acid sequence that is at least about 80%, at least about 85%, at least about 90%, or at least about 95% or greater sequence similar to SEQ ID NO:3.

The invention also provides transgenic plant parts comprising any of the DNA constructs of the invention, obtained from any of the transgenic food crop plants of the invention. Such parts are selected from the group consisting of a protoplast, a cell, a tissue, an organ, a cutting, and an explant, and include an inflorescence, a flower, a sepal, a petal, a pistil, a stigma, a style, an ovary, an ovule, an embryo, a receptacle, a seed, a fruit, a stamen, a filament, an anther, a male or female gametophyte, a pollen grain, a meristem, a terminal bud, an axillary bud, a leaf, a stem, a root, a tuberous root, a rhizome, a tuber, a stolon, a corm, a bulb, an offset, a cell of said plant in culture, a tissue of said plant in culture, an organ of said plant in culture, and a callus.

The invention also provides processed food or feed compositions obtained from any part of a fungal-resistant transgenic food crop plant of the invention, for example from a flower, a fruit, a stem, a leaf, a seed, a root, a tuber, or other edible plant part. The processed food or feed composition can be, for example, a meal, a flour, an oil, or a starch. In certain embodiments, mycotoxin levels in the food or feed composition of the invention are reduced by at least 50% compared that in processed food or feed compositions derived from otherwise identical, non-transgenic counterpart control plants. In other embodiments, mycotoxin levels in the food or feed composition of the invention are reduced by at least about 75%, at least about 80%, at least about 85%, at least about 90%, or at least about 95% or more compared to processed food or feed compositions derived from otherwise identical, non-transgenic counterpart control plants. The mycotoxin that is reduced in the food or feed compositions of the invention can be an aflatoxin, a fumonisin, a vomitoxin, or a trichothecene.

Also provided herein are methods of making a transgenic food crop plant of the invention. In certain embodiments, the method comprises: a) introducing any of the DNA constructs of the invention into a plant, plant cell, or plant tissue. Such DNA constructs can comprise a recombinant nucleic acid construct comprising a promoter operably linked to a nucleic acid encoding a heterologous signal peptide that is operably linked to a non-native nucleic acid encoding a KP6 antifungal protein, or the α and β polypeptides, operably linked to a polyadenylation sequence into a plant, a plant cell, or a plant tissue; and b) selecting for a transgenic food crop plant comprising the recombinant nucleic acid construct, wherein the transgenic food crop plant selected in step (b) expresses the KP6 antifungal protein, or the α and β polypeptides, and provide for at least about 50% inhibition of a plant pathogenic fungal infection compared to that in an otherwise identical counterpart control plant that lacks the recombinant nucleic acid construct. In some cases, accumulation in the apoplast is preferred.

The nucleic acid construct can be introduced into the plant, plant cell, or plant tissue in step (a) by any method known in the art, for example particle bombardment, DNA transfection, DNA electroporation, Agrobacterium-mediated, Rhizobium-mediated, and Sinorhizobium-mediated transformation. The nucleic acid construct can further comprise a sequence encoding a selectable marker, and the transgenic food crop plant is obtained in step (b) by growing the plant, plant cell, or plant tissue under conditions requiring expression of the selectable marker for plant growth.

The present methods provide for transgenic food crop plants that exhibit at least about 75%, at least about 80%, at least about 85%, at least about 90%, or at least about 95% or greater inhibition of a plant pathogenic fungal infection relative to that in an otherwise identical counterpart control plant that lacks a recombinant nucleic acid construct as disclosed herein.

### DEFINITIONS

Unless defined otherwise, all technical and scientific terms used herein have the same meaning as commonly understood by those of ordinary skill in the art to which the invention pertains. Any methods and materials similar or equivalent to those described herein can be used in the practice or testing of the present invention in place of the methods and materials described herein.

For the purposes of the present invention, the following terms are defined below.

The term “food crop plant” refers to plants that are either directly edible, or which produce edible products, and that are customarily used to feed humans either directly, or indirectly through animals. Non-limiting examples of such plants include:
1. Cereal crops: wheat, rice, maize (corn), barley, oats, sorghum, rye, and millet;
2. Protein crops: peanuts, chickpeas, lentils, kidney beans, soybeans, lima beans;
3. Roots and tubers: potatoes, sweet potatoes, and cassavas;
4. Oil crops: soybeans, corn, canola, peanuts, palm, coconuts, safflower, cottonseed, sunflower, flax, olive, and safflower;
5. Sugar crops: sugar cane and sugar beets;
6. Fruit crops: bananas, oranges, apples, pears, breadfruit, pineapples, and cherries;
7. Vegetable crops and tubers: tomatoes, lettuce, carrots, melons, asparagus, etc.
8. Nuts: cashews, peanuts, walnuts, pistachio nuts, almonds;
9. Forage and turf grasses;
10. Forage legumes: alfalfa, clover;
11. Drug crops: coffee, cocoa, kola nut, poppy;
12. Spice and flavoring crops: vanilla, sage, thyme, anise, saffron, menthol, peppermint, spearmint, coriander

Tobacco is explicitly excluded from the definition of “food crop plant” as used herein.

The articles “a” and “an” are used herein to refer to one or to more than one (i.e., to at least one) of the grammatical object of the article. By way of example, “an element” means one element or more than one element.

The singular terms “a”, “an”, and “the” include plural referents unless context clearly indicates otherwise. Similarly, the word “or” is intended to include “and” unless the context clearly indicates otherwise. Hence, comprising A or B means including A, or B, or A and B.

By “about” is meant a quantity, level, value, number, frequency, percentage, dimension, size, amount, weight, length, or the like, that varies by as much as ±30%, ±25%, ±20%, ±15%, ±10%, ±9%, ±8%, ±7%, ±6%, ±5%, ±4%, ±3%, ±2%, or ±1% compared to a reference quantity, level, value, number, frequency, percentage, dimension, size, amount, weight, length, or the like.

The term “comprising” as used in a claim herein is open-ended, and means that the claim must have all the features specifically recited therein, but that there is no bar on additional features that are not recited being present as well. The term “comprising” leaves the claim open for the inclusion of unspecified ingredients even in major amounts. The term “consisting essentially of” in a claim means that the invention necessarily includes the listed ingredients, and is open to unlisted ingredients that do not materially affect the basic and novel properties of the invention. A “consisting essentially of” claim occupies a middle ground between closed claims that are written in a closed “consisting of” format and fully open claims that are drafted in a “comprising′ format”. These terms can be used interchangeably herein if, and when, this may become necessary.

Furthermore, the use of the term “including”, as well as other related forms, such as “includes” and “included”, is not limiting.

The terms “antifungal protein” or “antifungal polypeptide” refer to proteins or polypeptides that exhibit any one or more of the following characteristics inhibiting or retarding the growth of fungal cells, killing fungal cells, inhibiting damage to a plant caused by fungal cells, inhibiting, disrupting, or retarding stages of the fungal life cycle such as spore germination, sporulation, or mating, and/or disrupting or inhibiting fungal cell infection, penetration, or spread within a plant.

The phrases “a plant pathogenic fungus inhibitory amount”, “antifungal effective amount”, or the like as used herein in the context of a transgenic food crop plant expressing a KP6 protein or α and β polypeptides refers to an amount of such KP6 protein or α and β polypeptides that results in any measurable decrease, i.e., at least about a 5% decrease, at least about a 10% decrease, at least about a 15% decrease, at least about a 20% decrease, at least about a 25% decrease, at least about a 30% decrease, at least about a 35% decrease, at least about a 40% decrease, at least about at 45% decrease, or at least about a 50% decrease or more in fungal growth or damage in a transgenic food crop plant of the present invention and/or any measurable decrease in the adverse effects caused by fungal growth in the transgenic food crop plant compared to that in an otherwise identical counterpart control non-transgenic plant exposed to the same fungus under the same conditions.

The terms “inhibit”, “inhibits”, “inhibiting”, and the like mean the ability to substantially antagonize, prohibit, prevent, restrain, slow, impede, repress, hinder, interfere with, disrupt, eliminate, stop, reduce, or reverse the biological effects of a fungal pathogen.

The phrase “inhibiting growth of a plant pathogenic fungus” or the like as used herein refers to methods that result in any measurable decrease, i.e., at least about a 10% decrease, at least about a 15% decrease, at least about a 20% decrease, at least about a 25% decrease, at least about a 30% decrease, at least about a 35% decrease, at least about a 40% decrease, at least about a 45% decrease, or at least about a 50% or greater decrease compared to that in an otherwise identical counterpart control non-transgenic plant exposed to the same fungus under the same conditions, in fungal growth, where fungal growth includes, but is not limited to, any measurable decrease in the numbers and/or extent of fungal cells, spores, conidia, or mycelia. As used herein, “inhibiting growth of a plant pathogenic fungus” and the like is also understood to include any measurable decrease, such as those enumerated above, in the adverse effects cause by fungal growth in a plant, including any type of plant tissue damage or necrosis, any type of plant yield reduction, any reduction in the value of the crop plant product, and/or production of undesirable fungal metabolites or fungal growth by-products including, but not limited to, mycotoxins.

The phrases “combating fungal damage”, “combating or controlling fungal damage”, “controlling fungal damage”, “inhibiting fungal damage”, or “resisting fungal damage” and the like as used herein in an agricultural context refer to reduction in damage to a crop due to infection by a fungal pathogen. In general, these phrases refer to reduction in the adverse effects caused by the presence of an undesired fungus in any particular locus. More particularly, these phrases refer to reduction, i.e., at least about a 10% decrease, at least about a 15% decrease, at least about a 20% decrease, at least about a 25% decrease, at least about a 30% decrease, at least about a 35% decrease, at least about a 45% decrease, or at least about a 50% or greater decrease in damage done to a transgenic food crop plant of the present invention by a fungal pathogen compared to that done to an otherwise identical, non-transgenic counterpart control plant by the same fungal pathogen under the same growth conditions. Adverse effects of fungal growth in or on plants include, but are not limited to, any type of plant tissue damage or necrosis, any type of plant yield reduction, any reduction in the value of the transgenic food crop plant product, and/or production of undesirable fungal metabolites or fungal growth by-products including, but not limited to, mycotoxins.

The term “treating” (or “treat” or “treatment”) means slowing, interrupting, arresting, controlling, stopping, reducing, or reversing the progression or severity of a symptom, disorder, condition, or disease caused by a plant fungal pathogen, and can include a total elimination of all fungal disease-related symptoms, conditions, or disorders of affected plants.

The term “structural coding sequence” refers to a DNA sequence that encodes a peptide, polypeptide, or protein that is made by a cell following transcription of the structural coding sequence to messenger RNA (mRNA), followed by translation of the mRNA to the desired peptide, polypeptide, or protein product.

The phrase “operably linked for expression” encompasses nucleic acid sequences linked in the 5′ to 3′ direction in such a way as to facilitate expression of an included nucleotide coding sequence.

A “promoter” is a DNA regulatory region capable of binding RNA polymerase in a cell and initiating transcription of a downstream (3′ direction) coding sequence.

As used herein the term “isolated” with reference to a nucleic acid molecule, polypeptide, or other biomolecule, means that the nucleic acid or polypeptide has separated from the genetic environment from which the polypeptide or nucleic acid were obtained. It can also mean altered from the natural state. For example, a polynucleotide or a polypeptide naturally present in a living animal is not “isolated” but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as an “isolated polypeptide” or an “isolated nucleic acid molecules” are polypeptides or nucleic acid molecules that have been purified, partially or substantially, from a recombinant host cell or from a native source.

The phrase “biological (or biologically) functional equivalents” and the like refers to peptides, polypeptides, and proteins that contain a sequence or structural feature similar to that within a KP6 protein or α or β polypeptide of the present invention, and that exhibit the same or similar, i.e., at least about 50%, at least about 75%, at least about 80%, at least about 85%, at least about 90%, at least about 95%, at least about 96%, at least about 97%, at least about 98%, or at least about 99% or more of the antifungal activity of a KP6 protein or α or β polypeptide of the present invention, in one of the antifungal assays described in the Examples herein. Thus, the present invention includes KP6 antifungal protein and α and β polypeptide analogs, derivatives, muteins, and variant allelic forms, and KP6 antifungal protein and α and β polypeptides containing conservative amino acid substitutions therein, that retain the same, or substantially the same, i.e., within about ±25%, antifungal activity of KP6 and the α and β polypeptides as that described herein.

In the present invention, sequence similarity or identity can be determined using the “BestFit” or “Gap” programs of the Sequence Analysis Software Package, Genetics Computer Group, Inc. University of Wisconsin Biotechnology Center, Madison, Wis. 53711.

Sequence identity refers to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys, and Met) occurs in both sequences to yield the number of matched positions, dividing the number of matched positions by the total number of positions in the window of comparison (i.e., the window size), and multiplying the result by 100 to yield the percentage of sequence identity.

Methods of alignment of sequences for comparison are well-known in the art. Optimal alignment of sequences for comparison may be conducted by the local homology algorithm of Smith and Waterman (1981) Adv. Appl. Math. 2: 482; by the homology alignment algorithm of Needleman and Wunsch (1970) J. Mol. Biol. 48: 443; by the search for similarity method of Pearson and Lipman (1988) Proc. Nat. Acad. Sci. 85: 2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the GCG® Wisconsin Package™ from Accelrys, Inc., San Diego, Calif.

The CLUSTAL program is well described by Higgins and Sharp (1988) Gene 73: 237-244; Higgins and Sharp (1989) CABIOS 5: 151-153; Corpet et al. (1988) Nucleic Acids Research 16:10881-90; Huang et al. (1992) Computer Applications in the Biosciences 8: 155-65; and Pearson et al. (1994) Methods in Molecular Biology 24: 307-331. A description of BLAST (Basic Local Alignment Search Tool) is provided by Altschul et al. (1993) J. Mol. Biol. 215:403-410.

Terms used to describe sequence relationships between two or more polynucleotides or polypeptides include “reference sequence,” “comparison window,” “sequence identity,” “percentage of sequence identity” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al. (1997) Nucl. Acids Res. 25:3389. A detailed discussion of sequence analysis can be found in Unit 19.3 of Ausubel et al. (1994-1998) Current Protocols in Molecular Biology, John Wiley & Sons Inc. Chapter 15.

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes).

In certain embodiments, the length of a reference sequence aligned for comparison purposes is at least 30%, preferably at least 40%, more preferably at least 50%, more preferably at least 60%, and even more preferably at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch algorithm ((1970) J. Mol. Biol. 48: 444-453) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frame shift gap penalty of 5.

The percent identity between two amino acid or nucleotide sequences can be determined using the algorithm of Meyers and Miller ((1989) Cabios 4:11-17) which has been incorporated into the ALIGN program (version 2.0), using a PAM120 weight residue table, a gap length penalty of 12, and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul et al. (1990) J. Mol. Biol. 215: 403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used.

Similarly, in particular embodiments of the invention, two amino acid sequences are “substantially homologous” or “substantially similar” when greater than 90% of the amino acid residues are identical. Two sequences are functionally identical when greater than about 95% of the amino acid residues are similar. Preferably the similar or homologous polypeptide sequences are identified by alignment using, for example, the GCG (Genetics Computer Group, Version 7, Madison, Wis.) pileup program, or using any of the programs and algorithms described above. The program may use the local homology algorithm of Smith and Waterman with the default values: Gap creation penalty=−(1+1/k), k being the gap extension number, Average match=1, Average mismatch=−0.333.

### Biologically Functionally Equivalent Polypeptides and Proteins

The present invention includes not only KP6 core protoxin (SEQ ID NO:5) and the α and β polypeptides (SEQ ID NOs:9 and 11, respectively), but also biologically functional equivalent proteins and polypeptides. The phrase “biologically functional equivalent proteins and polypeptides” denotes proteins and polypeptides that contain a sequence or moiety exhibiting sequence similarity to KP6 core protoxin (SEQ ID NO:5) and the α and β polypeptides (SEQ ID NOs:9 and 11, respectively), and which exhibit the same or similar, i.e., within about ±25%, antifungal activity as that of these molecules.

Proteins and Polypeptides Containing Conservative Amino Acid Changes in the KP6 Protein and α and β Polypeptide Sequences

It is well known that certain amino acids can be substituted for other amino acids in a protein structure without appreciable loss of biochemical or biological activity. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. Thus, various changes can be made in the amino acid sequences disclosed herein, or in the corresponding DNA sequences that encode these amino acid sequences without appreciable loss of their biological utility or activity.

Proteins and polypeptides biologically functionally equivalent to KP6 and the α and β polypeptides disclosed herein include amino acid sequences containing conservative amino acid changes in the fundamental sequences shown in SEQ ID NOs:5, 9, and 11, respectively. In such amino acid sequences, one or more amino acids in the fundamental sequence can be substituted, for example, with another amino acid(s), the charge and polarity of which is similar to that of the native amino acid, i.e., a conservative amino acid substitution, resulting in a silent change.

It should be noted that there are a number of different classification systems in the art that have been developed to describe the interchangeability of amino acids for one another within peptides, polypeptides, and proteins. The following discussion is merely illustrative of some of these systems, and the present invention encompasses any of the “conservative” amino acid changes that would be apparent to one of ordinary skill in the art of peptide, polypeptide, and protein chemistry from any of these different systems.

As disclosed in U.S. Pat. No. 5,599,686, certain amino acids in a biologically active peptide, polypeptide, or protein can be replaced by other homologous, isosteric, and/or isoelectronic amino acids, wherein the biological activity of the original molecule is conserved in the modified peptide, polypeptide, or protein. The following list of amino acid replacements is meant to be illustrative and is not limiting:

| Original | Replacement |
|---|---|
| Amino Acid | Amino Acid(s) |
| Ala | Gly |
| Arg | Lys, ornithine |
| Asn | Gln |
| Asp | Glu |
| Glu | Asp |
| Gln | Asn |
| Gly | Ala |
| Ile | Val, Leu, Met, Nle (norleucine) |
| Leu | Ile, Val, Met, Nle |
| Lys | Arg |
| Met | Leu, Ile, Nle, Val |
| Phe | Tyr, Trp |
| Ser | Thr |
| Thr | Ser |
| Trp | Phe, Tyr |
| Tyr | Phe, Trp |
| Val | Leu, Ile, Met, Nle |

In another system, substitutes for an amino acid within a fundamental sequence can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine. and glutamine; (4) neutral nonpolar (hydrophobic) amino acids such as alanine, leucine, isoleucine, valine, proline, phenylalanine, tryptophan, and methionine.

Conservative amino acid changes within a fundamental peptide, polypeptide, or protein sequence can be made by substituting one amino acid within one of these groups with another amino acid within the same group.

Some of the other systems for classifying conservative amino acid interchangeability in peptides, polypeptides, and proteins applicable to the sequences of the present invention include, for example, the following:

1. Functionally defining common properties between individual amino acids by analyzing the normalized frequencies of amino acid changes between corresponding proteins of homologous organisms (Schulz, G. E. and R. H. Schirmer (1979) Principles of Protein Structure (Springer Advanced Texts in Chemistry), Springer-Verlag). According to such analyses, groups of amino acids can be defined where amino acids within a group exchange preferentially with each other, and therefore resemble each other most in their impact on overall protein structure;

2. Making amino acid changes based on the hydropathic index of amino acids as described by Kyte and Doolittle (1982) J. Mol. Biol. 157(1):105-32. Certain amino acids can be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those that are within ±1 are particularly preferred, and those within ±0.5 are even more particularly preferred;

3. Substitution of like amino acids on the basis of hydrophilicity. U.S. Pat. No. 4,554,101 states that the greatest local average hydrophilicity of a protein, as governed by the hydrophilicity of its adjacent amino acids, correlates with a biological property of the protein. As detailed in this patent, the following hydrophilicity values have been assigned to amino acid residues: arginine (+3.0); lysine (+3.0); aspartate (+3.0.±0.1); glutamate (+3.0.±0.1); serine (+0.3); asparagine (+0.2); glutamine (+0.2); glycine (0); threonine (−0.4); proline (−0.5.±0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

4. Betts and Russell ((2003), “Amino Acid Properties and Consequences of Substitutions”, 0, Michael R. Barnes and Ian C. Gray, Eds., John Wiley & Sons, Ltd, Chapter 14, pp. 289-316) review the nature of mutations and the properties of amino acids in a variety of different protein contexts with the purpose of aiding in anticipating and interpreting the effect that a particular amino acid change will have on protein structure and function. The authors point out that features of proteins relevant to considering amino acid mutations include cellular environments, three-dimensional structure, and evolution, as well as the classifications of amino acids based on evolutionary, chemical, and structural principles, and the role for amino acids of different classes in protein structure and function in different contexts. The authors note that classification of amino acids into categories such as those shown in FIG. 14.3 of their review, which involves common physico-chemical properties, size, affinity for water (polar and non-polar; negative or positive charge), aromaticity and aliphaticity, hydrogen-bonding ability, propensity for sharply turning regions, etc., makes it clear that reliance on simple classifications can be dangerous, and suggests that alternative amino acids could be engineered into a protein at each position. Criteria for interpreting how a particular mutation might affect protein structure and function are summarized in section 14.7 of this review, and include first inquiring about the protein, and then about the particular amino acid substitution contemplated.

Biologically functional equivalents of KP6, or the α and β polypeptides, can have 10 or fewer conservative amino acid changes, more preferably seven or fewer conservative amino acid changes, and most preferably five or fewer conservative amino acid changes. The encoding nucleotide sequence (e.g., gene, plasmid DNA, cDNA, or synthetic DNA) will thus have corresponding base substitutions, permitting it to code for the biologically functionally equivalent form of KP6 or the α and β polypeptides. Due to the degeneracy of the genetic code, i.e., the existence of more than one codon for most of the amino acids naturally occurring in proteins, other DNA (and RNA) sequences that contain essentially the same genetic information as these nucleic acids, and which encode the same amino acid sequence as that encoded by these nucleic acids, can be used in practicing the present invention. This principle applies as well to any of the other nucleotide sequences disclosed herein.

The biologically functional equivalent proteins and polypeptides contemplated herein can possess about 70% or greater sequence similarity, more preferably about 80% or greater sequence similarity, more preferably about 90% or greater sequence similarity, and even more preferably about 95%, 96%, 97%, 98%, or 99% or greater sequence similarity to the sequence of, or corresponding moiety within, the fundamental core KP6 (SEQ ID NO:5) or α (SEQ ID NO:9) and β α (SEQ ID NO11) polypeptide amino acid sequence.

The biologically functional equivalent peptides, polypeptides, and proteins contemplated herein can possess about 70% or greater sequence identity, preferably about 85% or greater sequence identity, more preferably about 90% to about 95% sequence identity, and most preferably about 96%, 97%, 98%, or 99% sequence identity to the sequence of, or corresponding moiety within, the core KP6 protein or α and β polypeptide sequences.

Naturally Occurring Variants of KP6 and the α and β Polypeptides

Naturally occurring variants of KP6 and the α and β polypeptides having the “same or similar antifungal activity” (as defined above) as the molecules disclosed herein can be readily isolated using conventional DNA-DNA or DNA-RNA hybridization techniques, or by amplification using Polymerase Chain Reaction (PCR) methods. These variant forms should possess the ability to confer resistance to fungal pathogens when introduced by conventional transformation techniques into plants normally sensitive to such pathogens, or when introduced into plant-colonizing microorganisms to be applied to plants. Such resistance can be assayed by the methods described in the examples below.

Although embodiments of nucleotide sequences encoding KP6 and the α and β polypeptides are disclosed herein, it should be understood that the present invention also includes nucleotide sequences that hybridize to these sequences, and their complementary sequences, and that code on expression for proteins and polypeptides having the same or similar antifungal activity, as defined above, as that of KP6 and the α and β polypeptides. Such nucleotide sequences preferably hybridize to the present sequences, or their complementary sequences, under conditions of moderate to high stringency (see Green and Sambrook (2012) Molecular Cloning: A Laboratory Manual, Fourth Edition, Cold Spring Harbor Laboratory Press; Ausubel et al. (2003 and periodic supplements) Current Protocols in Molecular Biology, John Wiley & Sons, New York, N.Y.). Exemplary conditions include initial hybridization in 6×SSC, 5×Denhardt's solution, 100 μg ml fish sperm DNA, 0.1% SDS, at 55° C. for sufficient time to permit hybridization (e.g., several hours to overnight), followed by washing two times for 15 min each in 2×SSC, 0.1% SDS, at room temperature, and two times for 15 min each in 0.5-1×SSC, 0.1% SDS, at 55° C., followed by autoradiography. Typically, the nucleic acid molecule is capable of hybridizing when the hybridization mixture is washed at least one time in 0.1×SSC at 55° C., preferably at 60° C., and more preferably at 65° C.

The present invention also encompasses nucleotide sequences that hybridize to the sequences disclosed herein, and their complementary sequences, under salt and temperature conditions equivalent to those described above, and that code on expression for a protein or polypeptide that has the same or similar antifungal activity, as defined above, as that of KP6 and the α and β polypeptides disclosed herein. Such nucleotide sequences include oligonucleotide hybridization probes useful in screening genomic and other nucleic acid libraries for DNA sequences encoding polypeptides and proteins having antifungal activity the same or similar to that of KP6 and the α and β polypeptides are disclosed herein, which probes can be designed based on the sequences provided herein. Such probes can range from about 16 to about 28 nucleotides in length, generally about 16 nucleotides in length, more typically about 20 nucleotides in length, preferably about 24 nucleotides in length, and more preferably about 28 nucleotides in length. Preferably, these probes specifically hybridize to genomic RNA and DNA and other nucleic sequences encoding proteins or polypeptides having the same or similar antifungal activity, as defined above, as that of the α and β polypeptides disclosed herein. The antifungal activity thereof can be assessed by the methods disclosed in the examples herein. By such means proteins and polypeptides biologically functionally equivalent thereto, useful in controlling undesired fungi and protecting plants against fungal pathogens, can be isolated.

Polypeptides and Proteins that React with Antibodies Raised Against KP6 Antifungal Protein and the α and β Polypeptides

Biologically functional equivalent forms of KP6 and the α and β polypeptides also include proteins and polypeptides that react with, i.e., specifically bind to, antibodies raised against KP6 and the α and β polypeptides, and that exhibit the same or similar antifungal activity as these molecules, respectively. Such antibodies can be polyclonal or monoclonal antibodies. The “same or similar antifungal activity” can be about 70%, about 75%, about 80%, about 85%, about 90%, about 95%, about 96%, about 97%, about 98%, about 99%, or about 100% or greater than the antifungal activity of the KP6 antifungal protein or the α or β polypeptide.

Codon-Optimized, Synthetic DNA Sequences Designed for Enhanced Expression in Particular Host Cells

Biologically functional equivalent forms of the encoding nucleic acids of the present invention also include synthetic DNAs and RNAs designed for enhanced expression in particular host cells. Host cells often display a preferred pattern of codon usage (Murray et al. (1989) Nucl. Acids. Res. 17:477-498). Synthetic nucleic acids designed to enhance expression in a particular host should therefore reflect the pattern of codon usage in the host cell.

### Recombinant Methods

The present invention can be carried out using conventional techniques of chemistry, molecular biology, microbiology, recombinant DNA technology, and immunology, which are well known in the art and within the capabilities of a person of ordinary skill in the art. Such techniques are explained in the literature. See, for example, Green and Sambrook (2012) Molecular Cloning: A Laboratory Manual, Fourth Edition, Cold Spring Harbor Laboratory Press; Ausubel et al. (2003 and periodic supplements) Current Protocols in Molecular Biology, John Wiley & Sons, New York, N.Y.; B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, IRL Press; D. M. J. Lilley and J. E. Dahlberg, 1992, Methods in Enzymology: DNA Structure Part A: Synthesis and Physical Analysis of DNA, Academic Press; Buchanan et al., 2002, Biochemistry and Molecular Biology of Plants, Wiley; Miki and Iyer, Plant Metabolism, 2^{nd} Ed., D T Dennis, D H Turpin, D D Lefebrve, D G Layzell (eds.) Addison Wesley, Langgmans Ltd. London (1997); and Lab Ref: A Handbook of Recipes, Reagents, and Other Reference Tools for Use at the Bench, Edited by Jane Roskams and Linda Rodgers, 2002, Cold Spring Harbor Laboratory, ISBN 0-87969-630-3. The entire contents of each of these texts is herein incorporated by reference.

The methods of the present invention can be carried out in a variety of ways. The present antifungal peptides, polypeptides, and proteins, prepared by any of the methods described herein, can be applied directly to plants in a mixture with earners or other additives, including other antifungal agents, as is known in the art. Alternatively, the polypeptides can be expressed by bacterial or yeast cells that can be applied to plants. Plant cells can also be transformed by conventional means to contain DNA encoding the antifungal peptides, polypeptides, or proteins. These can be expressed constitutively, in a tissue-specific manner, or upon exposure of the plant to a fungal pathogen.

The present invention also encompasses the use of any of the DNA sequences or biologically functional equivalents thereof to produce recombinant plasmids, transformed microorganisms, probes, and primers useful in identifying related DNA sequences that confer resistance to fungal pathogens on plant cells, and to produce transgenic plants resistant to such fungal pathogens.

The present invention also encompasses methods of conferring resistance to fungal pathogens on plant cells and plants by using the DNA sequences or biologically functional equivalents thereof. Also encompassed by the present invention are genes encoding naturally occurring muteins and variants of KP6 that presumably exist in the genome of various endogenous, double-stranded RNA viruses in the cell cytoplasm of U. maydis strains, and which are capable of killing other susceptible strains of U. maydis and species of Fusarium. These genes can be isolated from the RNA of these viruses by conventional molecular biological methods.

DNA Constructs for Expression of KP6 and the α and β Polypeptides in Transgenic Plants

The present invention provides DNA constructs or expression vectors that facilitate the expression of the DNA sequences disclosed herein in higher plants and various microorganisms. As used herein, the terms “vector construct” or “expression vector” refer to assemblies of DNA fragments operatively linked in a functional manner that direct the expression of the DNA sequences discussed herein, as well as any additional sequence(s) or gene(s) of interest.

The expression of a plant or microbial structural coding sequence (gene, cDNA, synthetic DNA, or other DNA) which exists in double-stranded DNA form involves transcription of messenger RNA (mRNA) from one strand of the DNA by RNA polymerase and subsequent processing of the mRNA pnmary transcnpt inside the nucleus. This processing involves a 3′ non-translated region which adds polyadenylate nucleotides to the 3′ end of the mRNA.

Transcription of DNA into mRNA is regulated by a region of DNA referred to as the “promoter.”

Vectors useful in the present invention therefore include promoter elements operably linked to coding sequences of interest, and can also include 5′ non-translated leader sequences, 3′ non-translated regions, and one or more selectable markers. A variety of such markers are well known in the art.

Signal Peptides

Certain constructs of the present invention comprise a sequence encoding a heterologous signal peptide that allows for secretion of KP6 protein and α and β polypeptides from plant cells. Such signal peptide sequences can include synthetic, or naturally occurring, signal peptide sequences derived from other well characterized secreted proteins that are joined to the coding sequence of an expressed gene, and are removed post-translationally from the initial translation product.

In certain embodiments, the heterologous signal peptide sequences derived from Medicago defensin proteins (Hanks et al. (2005) Plant Mol. Biol. 58, 385-399) can be used. Examples of Medicago defensin protein signal peptides include, but are not limited to, signal peptides of MsDef1 (GenBank Accession No. AAV85437.1; SEQ ID NO:3); MtDef1.1 (GenBank Accession No AAQ91287.1); MsDef1.6 (GenBank Accession No AAV85432.1); MtDef4 (Sagaram et al. (2011) PLoS One. 6(4):e18550); and MtDef2.1 (GenBank Accession No AAQ91290.1). The MtDef4 signal peptide and sequences encoding the same are disclosed in US Patent Application Publication No. 20080201800. Another example of a useful heterologous signal peptide encoding sequence that can be used in monocots is the signal peptide derived from a barley cysteine endoproteinase gene (Koehler and Ho (1990) Plant Cell. 8:769-83). Another example of a useful heterologous signal peptide encoding sequence that can be used in dicots is the tobacco PR1b signal peptide. It is understood that this group of exemplary heterologous signal peptides is non-limiting and that one skilled in the art could employ other heterologous signal peptides that are not explicitly cited here in the practice of this invention.

In certain embodiments of the invention, the heterologous signal peptide in such chimeric constructs provides for secretion of the KP6 protein and α and β polypeptides to the apoplast.

In other embodiments of the invention, additional sequences encoding peptides that provide for the localization of a KP6 protein and α and β polypeptides in subcellular organelles can be operably linked to the nucleic acid sequences encoding the respective amino acid sequences of these molecules. KP6 protein and α and β polypeptides that are operably linked to a heterologous signal peptide are expected to enter the secretion pathway and can be retained by organelles such as the endoplasmic reticulum (ER) or targeted to the vacuole by operably linking the appropriate retention or targeting peptides to the C-terminus of the KP6 protein and α and β polypeptides. Examples of vacuolar targeting peptides include, but are not limited to, a CTPP vacuolar targeting signal from the barley lectin gene. Examples of ER targeting peptides include, but are not limited to, a peptide comprising a KDEL amino acid sequence. Localization of KP6 protein and α and β polypeptides in either the endoplasmic reticulum or the vacuole can provide for desirable properties such as increased expression in transgenic plants and/or increased efficacy in inhibiting fungal growth and damage in transgenic plants.

A non-limiting example of a synthetic nucleotide sequence for expression in plants is SEQ ID NO:7. The chimeric gene represented by SEQ ID NO:7 encodes a KP6 proprotein comprising a MsDef1 signal peptide (SEQ ID NO:3) operably linked to a mature KP6 core protein sequence (SEQ ID NO:5).

In certain embodiments, the KP6 core protein for use in any of the methods, plants, and plant colonizing microorganisms of the present invention can have at least about 90% to about 95%, about 96%, about 97%, about 98%, about 99%, or 100% sequence identity to SEQ ID NO:5. Similarly, the α and β polypeptides for use in any of the methods, plants, and plant colonizing microorganisms of the present invention can each have at least about 90% to about 95%, about 96%, about 97%, about 98%, about 99%, or 100% sequence identity to SEQ ID NO:9 and SEQ ID NO:11, respectively.

Promoters

A large number of promoters, including constitutive, inducible, repressible, tissue-specific, temporal, etc., promoters from a variety of different sources are well known in the art, and can be used to express DNA encoding KP6 and the α and β polypeptides in plant and microbial cells. Non-limiting examples of useful promoters active in plants include, for example, the nopaline synthase (nos) promoter, mannopme synthase (mas) promoter, and octopine synthase (ocs) promoters carried on tumor-inducing plasmids of Agrobacterium tumefaciens; the caulimovirus promoters such as the Cauliflower Mosaic Virus (CaMV) 19S or 35S promoter (U.S. Pat. No. 5,352,605) and CaMV 35S promoter with a duplicated enhancer (U.S. Pat. Nos. 5,164,316; 5,196,525; 5,322,938; 5,359,142; and 5,424,200); the Figwort Mosaic Virus (FMV) 35S promoter (U.S. Pat. No. 5,378,619); the cassava vein mosaic virus promoter (U.S. Pat. No. 7,601,885); the light-inducible promoter from the small subunit of ribulose-1,5-bisphosphate carboxylase (ssRUBISCO); the EIF-4A promoter from tobacco (Mandel et al. (1995) Plant Mol. Biol. 29: 995-1004); the chitinase promoter from Arabidopsis (Samac et al. (1991) Plant Cell 3: 1063-1072); the LTP (Lipid Transfer Protein) promoters from broccoli (Pyee et al. (1995) Plant J. 7: 49-59); the ubiquitin promoter from maize (Christensen et al. (1992) Plant Mol. Biol. 18:675-689); and the actin promoter from rice (McElroy et al. (1990) Plant Cell 2:163-171). All of these promoters have been used to create various types of DNA constructs that have been expressed in plants. Other useful promoters are described, for example, in U.S. Pat. Nos. 5,391,725; 5,428,147; 5,447,858; 5,608,144; 5,614,399; 5,633,441; 6,232,526; and 5,633,435, all of which are incorporated herein by reference.

Promoters active in certain plant tissues (i.e., tissue specific promoters) can be used to drive expression of KP6 and the α and β polypeptides. Expression of these molecules in the tissue that is typically infected by fungal pathogens is anticipated to be particularly useful. Thus, expression in reproductive tissues, seeds, roots, or leaves can be particularly useful in combating infection in those tissues by certain fungal pathogens in certain crops. Examples of useful tissue-specific, developmentally regulated promoters include, but are not limited to, the β-conglycinin 7S promoter, seed-specific promoters, and promoters associated with napin, phaseolin, zein, soybean trypsin inhibitor, ACP, stearoyl-ACP desaturase, or oleosin genes. Examples of root-specific promoters include, but are not limited to, the RB7 and RD2 promoters described in U.S. Pat. Nos. 5,459,252 and 5,837,876, respectively.

In the case of Fusarium infection, root-specific promoters are highly desirable.

There are at least 8 different root-specific promoters (Root 1-8) in soybean that can be used to target expression of KP6 to root tissue:

The GmRoot Promoter Family

| Name | Glyma# | Size |
|---|---|---|
| ROOT1 | Glyma12g07370.1 | 1541 |
| ROOT2 | Glyma15g02510.1 | 1531 |
| ROOT3 | Glyma11g36050.1 | 1411 |
| ROOT4 | Glyma10g31210.1 | 1450 |
| ROOT5 | Glyma12g29980.1 | 2557 |
| ROOT6 | Glyma07g12210.1 | 1251 |
| ROOT7 | Glyma20g36300.1 | 1492 |
| ROOT8 | Glyma12g08250.1 | 1434 |

Promoters useful in the present invention can also be selected based upon their ability to confer specific expression of a coding sequence in response to fungal infection. The infection of plants by fungal pathogens triggers the induction of a wide array of proteins, termed defense-related or pathogenesis-related (PR) proteins (Bowles (1990) Ann. Rev. Biochem. 59:873-907; Bol et al. (1990) Ann. Rev. Phytopathol. 28:113-138). Defense-related or PR genes have been isolated and characterized from a number of plant species. The promoters of these genes can be used to drive expression of KP6, the α and β polypeptides and biologically functional equivalents thereof in transgenic plants challenged with fungal pathogens. For example, such promoters have been derived from defense-related or PR genes isolated from potato plants (Fritzemeier et al. (1987) Plant Physiol. 85:34-41; Cuypers et al. (1988) Mol. Plant-Microbe Interact. 1:157-160; Logemann et al. (1989) Plant Cell 1:151-158; Matton et al. (1989) Mol. Plant-Microbe Interact. 2:325-331; Schroder et al. (1992) Plant J. 2:161-172). Alternatively, pathogen-inducible promoters such as the PRP1 promoter obtainable from tobacco (Martini et al. (1993) Mol. Gen. Genet. 263:179) can be employed.

Other useful promoters induced by fungal infections include those promoters associated with genes involved in phenylpropanoid metabolism (e.g., phenylalanine ammonia lyase, chalcone synthase promoters), genes that modify plant cell walls (e.g., hydroxyproline-rich glycoprotein, glycine-rich protein, and peroxidase promoters), genes encoding enzymes that degrade fungal cell walls (e.g., chitinase or glucanase promoters), genes encoding thaumatin-like protein promoters, or genes encoding proteins of unknown function that display significant induction upon fungal infection. Maize and Flax promoters, designated as Mis1 and Fis1, respectively, are also induced by fungal infections in plants and can be used (U.S. Patent Application 20020115849).

Additional useful promoters are those that are induced by various environmental stimuli including, but not limited to, promoters induced by heat (e.g., heat shock promoters such as Hsp70), promoters induced by light (e.g., the light-inducible promoter from the small subunit of ribulose 1,5-bisphosphate carboxylase, ssRUBISCO), promoters induced by cold (e.g., COR promoters), promoters induced by oxidative stress (e.g., catalase promoters), promoters induced by drought (e.g., the wheat Em and rice rab16A promoters), and promoters induced by multiple environmental signals (e.g., rd29A promoters, Glutathione-S-transferase (GST) promoters).

In any case, the particular promoter selected to drive the expression of KP6 and the α and β polypeptides in transgenic plants should be capable of causing sufficient expression of the coding sequences of these molecules to result in the production of an antifungal effective amount of KP6 and the α and β polypeptides in plant tissues. Examples of promoters capable of driving constitutive expression throughout plant development and throughout the plant are the CaMV35S, FMV35S, rice actin, maize ubiquitin, and eIF-4A promoters. Enhanced or duplicate versions of the CaMV35S and FMV35S promoters are particularly useful (U.S. Pat. No. 5,378,619).

It should be understood that the foregoing lists of promoters are exemplary and non-limiting, and that one skilled in the art could employ other promoters that are not explicitly cited here in the practice of this invention.

5′ Non-Translated Leader Sequences

The RNA produced by DNA constructs of the present invention can also contain a 5′ non-translated leader sequence. This sequence can be derived from the promoter selected to express the gene, and can be specifically modified so as to increase translation of the mRNA. The 5′ non-translated regions cam also be obtained from viral RNAs, from suitable eukaryotic genes, or from a synthetic gene sequence. However, the present invention is not limited to constructs wherein the 5′ non-translated region is derived from the 5′ non-translated sequence that accompanies the promoter sequence. Rather, the non-translated leader sequence can be derived from an unrelated promoter or coding sequence. For example, the petunia heat shock protein 70 (Hsp70) contains such a leader (Winter (1988) Mol. Gen. Genet. 221:315-319).

3′ Non-Translated Regions

The 3′ non-translated region of the chimeric constructs of the present invention can contain a transcriptional terminator, or an element having equivalent function, and a polyadenylation signal that functions in plants to cause the addition of adenylate nucleotides to the 3′ end of the mRNA. Examples of such 3′ regions include the 3′ transcribed, nontranslated regions containing the polyadenylation signal of Agrobacterium tumor-inducing (Ti) plasmid genes, such as the nopaline synthase (nos) gene, and plant genes such as the soybean 7s storage protein gene and pea ssRUBISCO E9 gene (Fischoff et al., European Patent Application 0 385 962).

All the foregoing elements can be combined to provide a recombinant, double-stranded DNA molecule, comprising operatively linked in the 5′ to 3′ direction:

a) a promoter that functions in plant cells to cause the production of an RNA sequence (transcript);

b) a DNA coding sequence that encodes KP6 or the α and β polypeptides; and

c) a 3′ non-translated region that functions in plant cells to cause transcnptional termination and the addition of polyadenylate nucleotides to the 3′ end of said RNA sequence.

The KP6 or α and β polypeptide DNA coding sequences can comprise the entire nucleotide sequences shown in SEQ ID NOs:7, 9, or 10. In the case of SEQ ID NO:7, KP6 will be transported to the extracellular space. In the case of SEQ ID NOs:9 and 10, the α and β polypeptides can be targeted to the apoplast via the use of an apoplast targeting sequence such as the MsDef1 export signal sequence (SEQ ID NO:3). In all these cases, KP6 or the α and β polypeptides will be effective in controlling fungal damage.

Selectable Marker Genes

Selectable marker genes for selection of transformed cells or tissues can be included in transformation vectors. These can include genes that confer antibiotic resistance or resistance to herbicides. Examples of suitable selectable marker genes include, but are not limited to, genes encoding resistance to chloramphenicol (Herrera Estrella et al. (1983) EMBO J. 2:987-992); methotrexate (Herrera Estrella et al. (1983) Nature 303:209-213; Meijer et al. (1991) Plant Mol. Biol. 16:807-820); hygromycin (Waldron et al. (1985) Plant Mol. Biol. 5:103-108; Zhijian et al. (1995) Plant Science 108:219-227); streptomycin (Jones et al. (1987) Mol. Gen. Genet. 210:86-91); spectinomycin (Bretagne-Sagnard et al. (1996) Transgenic Res. 5:131-137); bleomycin (Hille et al. (1990) Plant Mol. Biol. 7:171-176); sulfonamide (Guerineau et al. (1990) Plant Mol. Biol. 15:127-136); bromoxynil (Stalker et al. (1988) Science 242:419423); glyphosate (Shaw et al. (1986) Science 233:478-481); and phosphinothricin (DeBlock et al. (1987) EMBO J. 6:2513-2518).

Introduction of DNA Constructs into Plants

DNA constructs of the present invention can be introduced into the genome of the desired plant host by a variety of conventional techniques including, for example, electroporation and microinjection of plant cell protoplasts; biolistic methods, such as DNA micro-particle bombardment; Agrobacterium tumefaciens-mediated infection; Rhizobium-mediated transformation; Sinorhizobium-mediated transformation; polyethylene glycol precipitation; DNA transfection; and “whiskers”-mediated transformation. Methods of integrating DNA molecules at specific locations in the genomes of transgenic plants through use of site-specific recombinases can also be used (U.S. Pat. No. 7,102,055).

### Regeneration of Transformed Plants

Methods of regeneration of transformed plants from protoplasts, cells, callus tissue, etc., are well known in the art. Note, for example, Bhojwani et al. (1996) Plant Tissue Culture: Theory and Practice (Revised ed.), Elsevier, ISBN 0-444-81623-2; George, Edwin F.; Hall, Michael A.; De Klerk, Geert-Jan, Eds. (2008) Plant Propagation By Tissue Culture. Volume 1. The Background (3rd ed.), Dordrecht: Springer, ISBN 978-1-4020-5004-6; and Singh and Srivastava (2006) Plant Tissue Culture, Campus Book International, ISBN 978-81-8030-123-0.

### Plant-Colonizing Microorganisms

U.S. Pat. No. 5,229,112 discloses a variety of plant-colonizing microorganisms, and methods of use, applicable to the core KP6 antifungal protein (SEQ ID NO: 5) and/or the α and β polypeptides (SEQ ID NOs:9 and 11, respectively).

The term “plant-colonizing microorganism” is used herein to refer to a microorganism that is capable of colonizing the “plant environment”, and which can express the core KP6 antifungal protein (SEQ ID NO:5) and/or the α and β polypeptides (SEQ ID NOs:9 and 11, respectively) in the “plant environment”. The plant colonizing microorganism is one that can exist in symbiotic or non-detrimental relationship with the plant in the plant environment. As used herein, the term “plant-colonizing microorganism” includes spore forming organisms of the family Bacillaceae, for example, Bacillus thuringiensis, Bacillus israelensis, and Bacillus subtilis.

The term “plant environment” refers to the surface of the plant, e.g., leaf, stem, buds, stalk, floral parts, or root surface, the interior of the plant and its cells, and to the “rhizosphere”, i.e., the soil which surrounds and which is influenced by the roots of the plant. Exemplary of the plant-colonizing microorganisms which can be engineered as taught herein are bacteria from the genera Pseudomonas, Agrobacterium, Rhizobium, Erwinia, Azotobacter, Azospirillum, Klebsiella, Alcaligenes and Flavobacterium. Rhizosphere-colonizing bacteria from the genus Pseudomonas are preferred for use herein, especially the fluorescent pseudomonads, e.g., Pseudomonas fluorescens, which is especially competitive in the plant rhizosphere and in colonizing the surface of the plant roots in large numbers. Another group of particularly suitable plant-colonizing microorganisms for use herein are those of the genus Agrobacterium; A. radiobacter may be particularly suitable. Examples of suitable phylloplane colonizing bacteria are P. putida, P. syringae, and Erwinna species.

The antifungal plant-colonizing microorganisms of the invention can be applied directly to the plant environment, e.g., to the surface of the leaves, buds, roots or floral parts, to the plant seed, or to the soil. When used as a seed coating, the plant-colonizing microorganisms of the invention are applied to the plant seed prior to planting. Generally, small amounts of the antifungally active microorganism will be required to treat such seeds.

The determination of an antifungal effective amount of plant-colonizing microorganisms useful in the methods of the present invention required for a particular plant is within the skill of the art, and will depend on such factors as the plant species, the fungal pathogen, method of planting, and the soil type, (e.g., pH, organic matter content, moisture content).

Theoretically, a single plant-colonizing microorganism of the invention containing DNA encoding the core KP6 antifungal protein (SEQ ID NO:5) and/or the α and β polypeptides (SEQ ID NOs:9 and 11, respectively) is sufficient to control fungal pathogens because it can grow into a colony of clones of sufficient number to express antifungal amounts of toxin. However, in practice, due to varying environmental factors which may affect the survival and propagation of the microorganism, a sufficient number of plant colonizing microorganisms should be provided in the plant environment (e.g., roots or foliage) to assure survival and/or proliferation. For example, application of 10³ to 10¹⁰ bacteria or yeasts per seed may be sufficient to insure colonization on the surface of the roots by the microorganism. It is preferred to dose the plant environment with enough bacteria or other plant-colonizing microorganism to maintain a population that expresses 50 to 250 nanograms of toxin. For example, 10⁵ to 10⁸ bacteria per square centimeter of plant surface may be adequate to control fungal infection. At least 0.5 nanograms, preferably 1 to 100 nanograms, of anti-fungal active protein or polypeptides may be sufficient to control fungal damage to plants.

Compositions containing the antifungally active plant-associated microorganisms of the invention can be prepared by formulating the biologically active microorganism with adjuvants, diluents, carriers, etc., to provide compositions in the form of finely-divided particulate solids, granules, pellets, wettable powders, dusts, aqueous suspensions, dispersions, or emulsions. Illustrative of suitable carrier vehicles are: solvents, e.g., water or organic solvents, and finely divided solids, e.g., kaolin, chalk, calcium carbonate, talc, silicates, and gypsum.

The present invention also encompasses the use of the antifungal plant-colonizing microorganisms in the methods and compositions of the invention in encapsulated form, e.g., the plant-colonizing microorganisms can be encapsulated within shell walls of polymer, gelatin, lipid, and the like. Other formulation aids such as, for example, emulsifiers, dispersants, surfactants, wetting agents, anti-foam agents, and anti-freeze agents, can be incorporated into the antifungal compositions, especially if such compositions will be stored for any period of time prior to use.

In addition to the antifungally active plant-colonizing microorganisms, the compositions of the invention can additionally contain other known biologically active agents, such as, for example, a herbicide, fungicide, or other insecticide. Also, two or more antifungally active plant-colonizing microorganisms can be combined.

The application of antifungal compositions containing the genetically engineered plant-colonizing microorganisms of the invention as the active agent can be carried out by conventional techniques utilizing, for example, spreaders, power dusters, boom and hand sprayers, spry dusters, and granular applicators.

The compositions of the invention are applied in an antifungally effective amount, which will vary depending on such factors as, for example, the specific fungal pathogen to be controlled, the specific plant (and plant part or soil) to be treated, and the method of applying the antifungally active compositions.

Plant-colonizing microorganisms expressing the core KP6 antifungal protein (SEQ ID NO:5) and/or the α and β polypeptides (SEQ ID NOs:9 and 11, respectively) useful in inhibiting fungal infection and damage in plants according to the present invention include, for example, bacteria selected from the group consisting of genera selected from Pseudomonas, Agrobacterium, Rhizobium, Erwinia, Azotobacter, Azospirillum, Klebsiella, Alcaligenes, and Flavobacterium, and yeasts selected from the group consisting of Saccharomyces cerevisiae, Pichia pastoris, and Pichia methanolica.

### Pharmaceutical and Agricultural Antifungal Compositions

The present invention not only encompasses transgenic plants expressing KP6 or the α and β polypeptides, transformed microorganisms that can be applied to the loci of plants, but also pharmaceutical and agricultural antifungal compositions that can be used for inhibiting the growth of, or killing, pathogenic fungi. These compositions can be formulated by conventional methods.

Pharmaceutical antifungal compositions comprising KP6 or the α and β polypeptides can be formulated by methods described in Remington: The Science and Practice of Pharmacy (2005), 21^{st} Edition, University of the Sciences in Philadelphia, Lippincott Williams & Wilkins. Such compositions can contain KP6 antifungal protein and/or a combination of the α and β polypeptides at concentration in the range of from about 0.1 μg ml to about 100 mg ml, preferably between about 5 μg ml and about 5 mg ml, at a pH in the range of from about 3.0 to about 9.0. The KP6 antifungal protein and/or the α and β polypeptides can be formulated alone, or in combination with other conventional antifungal compounds such as, by way of non-limiting example, polyene antifungals; imidazole, triazole, and thiazole antifungals; allylamines; and echinocandins.

Agricultural compositions can be formulated as described in, for example, Winnacker-Kuchler (1986) Chemical Technology, Fourth Edition, Volume 7, Hanser Verlag, Munich; van Falkenberg (1972-1973) Pesticide Formulations, Second Edition, Marcel Dekker, N.Y.; and K. Martens (1979) Spray Drying Handbook, Third Edition, G. Goodwin, Ltd., London. Necessary formulation aids, such as carriers, inert materials, surfactants, solvents, and other additives are also well known in the art, and are described, for example, in Watkins, Handbook of Insecticide Dust Diluents and Carriers, Second Edition, Darland Books, Caldwell, N.J., and Winnacker-Kuchler (1986) Chemical Technology, Fourth Edition, Volume 7, Hanser Verlag, Munich. Using these formulations, it is also possible to prepare mixtures of the present KP6 antifungal protein, and α and β polypeptides, with other pesticidally active substances, fertilizers, and/or growth regulators, etc., in the form of finished formulations or tank mixes.

As noted above, antifungal compositions contemplated herein also include those in the form of host cells, such as bacterial and fungal cells, capable of the producing the present KP6 antifungal protein and/or the α and β polypeptides, and which can colonize plants, including roots and/or leaves. Examples of bacterial cells that can be used in this manner include strains of Agrobacterium, Arthrobacter, Azospyrillum, Clavibacter, Escherichia, Pseudomonas, Rhizobacterium, and the like.

Numerous conventional fungal antibiotics and chemical fungicides with which the present KP6 antifungal protein and α and β polypeptides can be combined are known in the art, and are described in Worthington and Walker (1983) The Pesticide Manual, Seventh Edition, British Crop Protection Council. These include, for example, polyoxines, nikkomycines, carboxy amides, aromatic carbohydrates, carboxines, morpholines, inhibitors of sterol biosynthesis, and organophosphorus compounds. Other active ingredients which can be formulated in combination with the present antifungal polypeptide include, for example, insecticides, attractants, sterilizing agents, acancides, nematocides, and herbicides. U.S. Pat. No. 5,421,839 contains a comprehensive summary of the many active agents with which substances such as the present antifungal KP6 antifungal protein and α and β polypeptides can be formulated.

Whether alone or in combination with other active agents, the present antifungal KP6 protein and α and β polypeptides can be applied at a concentration in the range of from about 0.1 μg ml to about 100 mg ml, preferably between about 5 μg ml and about 5 mg ml, at a pH in the range of from about 3.0 to about 9.0. Such compositions can be buffered using, for example, phosphate buffers between about 1 mM and 1 M, preferably between about 10 mM and 100 mM, more preferably between about 15 mM and 50 mM. In the case of low buffer concentrations, it is desirable to add a salt to increase the ionic strength, preferably NaCl in the range of from about 1 mM to about 1 M, more preferably about 10 mM to about 100 mM.

At this time, transgenic soybean and wheat lines have been created that express active KP6 as demonstrated by Ustilago maydis killing assays (Example 4). These results demonstrate that the protoxin form of KP6 is properly processed into an active two-subunit antifungal composition by the plant cellular proteases. The biological activity of KP6 appears to be significantly higher than that observed in soybeans expressing KP4. KP4 is disclosed in the inventors' PCT International Publication WO 2012/012480. It should be noted that there is no sequence homology between the single subunit antifungal protein KP4 and KP6 antifungal protein. Furthermore, current understanding of the modes of action of KP4 and KP6 suggests no overlap in function.

The present invention discloses several transgenic lines of soybean and wheat that express KP6 antifungal protein that is normally produced by a Totivirus which persistently infects corn smut, Ustilago maydis, and which is secreted by the host. In the fungus, it is translated as a single polypeptide, and processed by Kex2p protease. In the experiments described below, the fungal export signal sequence at the N-terminus (SEQ ID NO:2) is removed and replaced with that from the plant defensin MsDef1 (SEQ ID NO:3). The gene is codon-optimized for expression in soybean and wheat, and made synthetically. As shown below, the chimeric KP6 gene introduced into soybean produces biologically active protein as determined by its ability to kill Ustilago maydis (Example 4) and other fungi, including Fusarium (Examples 5-8). Using this construct, one can generate transgenic wheat and maize.

The following examples are meant to be illustrative, and not limiting, of the practice and products of the present invention.

### Example 1

### Construction of Vector AKK/FMV/KP6

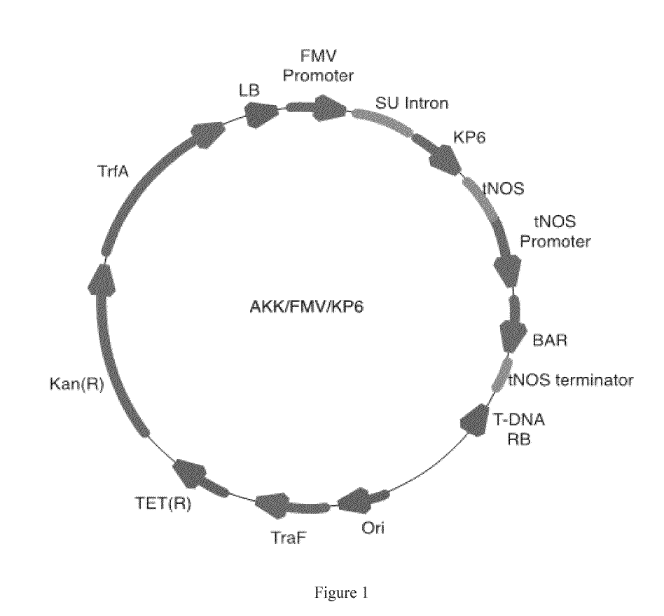

As shown in FIG. 1, the KP6 signal peptide sequence (SEQ ID NO:2) is replaced with the 27-amino acid secretory signal peptide sequence of a plant defensin, MsDef1 (SEQ ID NO:3). This signal peptide was previously shown to facilitate transport of proteins to the apoplast in transgenic plants (Allen et al. (2011) Plant Biotech Journal 9:857-864). In order to obtain a high level expression of KP6 in all organs of transgenic soybean, the chimeric KP6 gene is chemically synthesized using the native KP6 gene sequence (SEQ ID NO:1) and cloned as a Nco I-Xba I fragment between the Figwort mosaic virus 35S promoter (Sanger et al. (1990) Plant Molecular Biology 14:433-443) and nopaline synthase polyadenylation signal (Gleave (1992) Plant Molecular Biology 20:1203-1207) in the soybean expression vector AKK1472 (Hammes et al. (2005) Molecular Plant Microbe Interactions 18:1247-1257). The AKK1472 vector containing the KP6 chimeric gene and bar gene conferring Basta® resistance as a selectable marker gene (Thompson et al. (1985) EMBO Journal 6:2519-2523) is transferred to Agrobacterium tumefaciens strain EHA105 for soybean transformation (Clemente et al. (2000) Crop Sci. 40:797-803; Zhang et al. (1999) Plant Cell, Tiss. Organ Cult. 56:37-46).

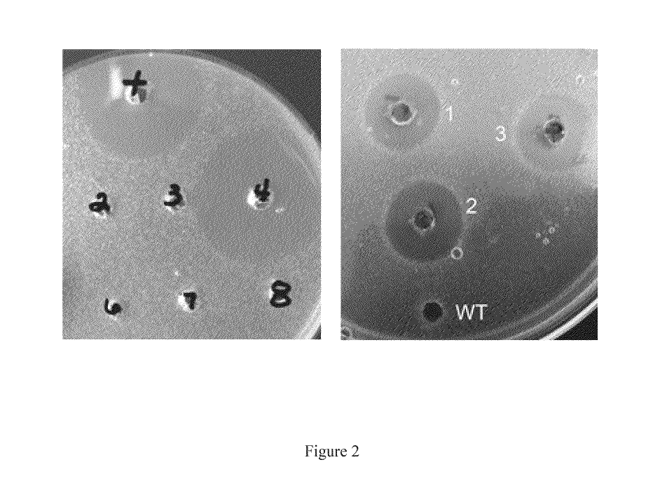

While vector AKK/FMV/KP6 is not optimized for expression of genes in monocots, the results shown in FIG. 2 (Example 4) demonstrate that it yields sufficient expression in wheat.

### Example 2

### Soybean Transformation and Regeneration of Transgenic Plants

The transformation protocol used in this example to create transgenic soybean lines using Agrobacterium has been previously described (Clemente et al. (2000) Crop Sci. 40:797-803; Zhang et al. (1999) Plant Cell, Tiss. Organ Cult. 56:37-46).

The exterior of the seeds (in this case the soybean variety called “Jack”) are first sterilized using commercial grade Clorox® (5% A aqueous sodium hypochlorite, NaClO) overnight. The sterilized seeds are then allowed to germinate in germination medium (GM; Gamborg's B5 medium (Gamborg et al. (1968) Experimental Cell Research 50:151) supplemented with 2% sucrose, pH 5.8, solidified with 0.8% agar) for 5 days at 24° C. (18/6) light regime). The A. tumefaciens transformed with the vector of Example 1 are collected via low speed centrifugation and suspended in co-cultivation medium to a final OD₆₅₀ of 0.6 to 1.0. Co-cultivation medium is 1/10th Gamborg's B5 medium supplemented with 1.67 mg/l 6-b enzylaminopurine (BAP), 0.25 mg/l gibberellic acid (GA3), 3% sucrose, 200 μM acetosyringone, 20 mM 2-(N-morpholino)-ethanesulfonic acid (MES), pH 5.4.

The following protocol has been previously described (Clemente et al. (2000) supra; Zhang et al. (1999) supra).

Agrobacterium inoculum is placed in a petri plate with the prepared explants (from wounded, germinating seed) for 30 min., with occasional agitation. The explants are then placed on co-cultivation plates (Petri dishes containing 0.76 g Gamborg Basal Salt Mixture, 7.4 g MES, 60 g sucrose, pH adjusted to 5.4 using 1 M KOH, and 5 g/L agarose dissolved in warm media), adaxial side down. The plates are wrapped with parafilm and place at 24° C., 18/6 light regime for 3 days. Following the co-cultivation period, the explants are briefly washed in liquid shoot initiation medium (3.08 g of Gamborgs B5 salts, 30 g Sucrose, 0.56 g MES, adjusted to pH to 5.6 using 1 M KOH) supplemented with 0.25 mg/l GA3. After the first two weeks, the hypocotyl region is cut flush to the developing node, and incubated for two weeks in the absence of glufosinate. The tissue is then transferred to fresh shoot initiation medium every two weeks, for a total of ˜10 weeks with 3 mg/l glufosinate. The tissue is oriented so that the freshly cut surface is imbedded in the medium, with the differentiating region flush to the surface. At the end of the shoot initiation period, only the differentiating explants are used. The cotyledons are removed from the explants, and a fresh cut is made at the base of the developing node (horizontally), the tissue is transferred to shoot elongation medium, and is cultured at 24° C. with a 18/6 light regime. Shoot elongation medium is composed of MS salts/Gamborg's vitamins supplemented with 1 mg/l zeatin riboside, 0.1 mg/l indole acetic acid (IAA), 0.5 mg/l GA3, 3% sucrose, 100 mg/l pyroglutamic acid, 50 mg/l asparagine, 3 mM MES (pH 5.6), solidified with 0.8% purified agar. Since the bar gene is used as a marker, 3 mg/l glufosinate is added. The tissue is transferred to fresh shoot elongation medium every two weeks. At each transfer, a fresh horizontal slice is made at the base of the tissue. Elongated shoots (>3 cm) are rooted on rooting medium without further selection. Rooting medium is composed of 4.33 g of Murashige & Skoog Basal Salt Mixture, 20 g sucrose, 0.56 g MES. The pH is adjusted to 5.6 using 1 M KOH and 3 g Phytagel per liter are added. The solution is autoclaved (20 min.) and when cooled, 1 ml Gamborg B5 vitamins (1000×), 1 ml L-asparagine monohydrate (50 mg/ml stock), and 1 ml L-Pyroglutamic acid (100 mg/ml stock) are added.

The plants are then grown and selected for using PCR to detect the presence of the KP6 gene and using the U. maydis killing assay described in Gu et al. (1995) Structure 3:805-814. This assay is essentially a suspension of the P2 strain of U. maydis in 4% agar. Wells are cut into the agar, and pieces of plant material are placed in each well. The presence of active antifungal proteins is made evident by killing rings around each sample (FIG. 2; Example 4). Homozygotes are eventually selected using quantitative PCR using the Promega GoTaq® qPCR master mix (Promega Corporation, Madison, Wis.) on an AB StepOne Plus-Real time PCR system (Applied Biosystems, Carlsbad, Calif.) per the manufacturer's instructions.

### Example 3

### Wheat Transformation and Regeneration of Transgenic Plants

The protocol used for wheat transformation was previously described by Cheng et al. (1997) Plant Physiology 115:971-980.

For these transformations, Triticum aestivum cv Bobwhite, is used. Immature caryopses are collected from plants 14 days after anthesis. Immature embryos are dissected aseptically and cultured on a semisolid or liquid CM4 medium (Zhou et al. (1995) Plant Cell Replication 15:159-163) with 100 mg L-ascorbic acid (CM4C). The MS salts (Murashige and Skoog (1962) Physiology Plant. 15:473-497) in the CM4 medium are adjusted to full strength (the original amounts) or one-tenth-strength (Fry et al. (1987) Plant Cell Reports 6: 321-325). The immature embryos are cultured for 3 to 4 hours. Embryogenic calli are prepared by culturing the immature embryos on CM4C medium for 10 to 25 days. The callus pieces derived from immature embryos are inoculated with A. tumefaciens using the embryogenic callus sectors. A. tumefaciens C58 (ABI) harboring the vector described in Example 1 (FIG. 1) is prepared as described above in Example 1 for soybean transformation. The A. tumefaciens is grown to a cell density of A₆₀₀ of 1 to 2 for inoculation. The immature embryos and embryogenic calli maintained on the CM4C medium are transferred into an A. tumefaciens cell suspension in Petri dishes. The inoculation is conducted at 23 to 25° C. for 3 hours in the dark. After inoculation, the A. tumefaciens cells are removed and the explants are placed on semisolid medium (Gelrite) with liquid CM4C with full-strength MS salts and supplemented with 10 g/L glucose and 200 μM acetosyringone. The co-cultivation is performed at 24 to 26° C. in the dark for 2 or 3 days. After co-culture, the infected immature embryos and calli are cultured on the solid CM4C medium with 250 mg/L carbenicillin for 2 to 5 days without selection. A. tumefaciens infected explants are then transferred to CM4C medium supplemented with 3 mg/l glufosinate and 250 mg/L carbenicillin for callus induction. Two weeks later, the explants are transferred to the first regeneration medium, MMS0.2C (consisting of MS salts and vitamins, 1.95 g/L MES, 0.2 mg/L 2,4-dichloro-phenoxyacetic acid, 100 mg/L ascorbic acid, and 40 g/L maltose, solidified by 2 g/L gelrite supplemented with 3 mg/l glufosinate and 250 mg/L carbenicillin. At transfer to the regeneration medium, each piece of callus derived from one immature embryo or one piece of inoculated callus is divided into several small pieces (approximately 2 mm). In another 2 weeks, young shoots and viable callus tissues are transferred to the second regeneration medium, MMSOC, which contains the same components as MMS.2C, with all antibiotics included. When the shoots develop into about 3 cm or longer plantlets, they are transferred to larger culture vessels containing the regeneration medium for further growth and selection. Leaf samples are taken from some of the plantlets for the U. maydis killing assay and PCR testing at this stage. Plants that are highly glufosinate resistant are transferred to soil. All of the plants derived from the same embryo or piece of callus are considered to be clones of a given event.

### Example 4

### Expression of Active KP6 in Wheat and Soybean

FIG. 2 shows that KP6 can be expressed in an active form in both wheat (left image) and in soybean (right image).

As described above (Example 2), this assay is performed by suspending the P2 strain of U. maydis in agar and placing portions of the transgenic plants in wells. Active antifungal proteins are denoted by clearing zones around the test wells. The ‘+’ mark on the left image denotes the application of purified KP6 protein and the ‘WT’ on the right image denotes the placement of the original, non-transgenic ‘Jack’ variety of soybean. The numbers in each panel indicate different transformation events.

These results demonstrate that both wheat and soybean can be transformed to express KP6 active against U. maydis.

### Example 5

### Effect of KP6 on the Growth of Various Fungi

The prevailing thought in the field has been that the U. maydis killer proteins are only effective against Ustilago.

To test this, purified KP6 is added to a number of fungi grown in liquid culture using the method of Spelbrink et al. (2004) Plant Physiology 135:2055-2067. Since these are all filamentous fungi, growth inhibition is qualitatively estimated by changes in hyphae mass.

While not an exhaustive survey, the results shown in Table 1 demonstrate that KP6 has significant antifungal activity against a number of different fungi other than U. maydis.

TABLE 1
Antifungal Activity of KP6 Against Various Fungi

| Antifungal Protein | Target | ED₅₀ | Inhibition |
|---|---|---|---|
| KP6 | Fusarium | 1 μM | 40% |
| | graminearum | | |
| KP6 | Neurospora crassa | 1 μM | 80% |
| KP6 | Fusarium | 2.5 μM | 20% |
| | verticillioides | | |
| KP6 | Aspergillus flavus | NA | NA |

NA = no activity.
Values are approximate.
ED₅₀ is the concentration of KP6 protein required to produce half maximal growth inhibition.

### Example 6

### Experiment 1

### Greenhouse Testing of KP6 Transgenic Soybean Lines Using a High Dose of Fusarium verguliforme on Soybean Plantlets

In this experiment, resistance of KP6 transgenic soybean lines infected with a high dose of Fusarium verguliforme is tested in the greenhouse. When performing the greenhouse challenges with F. virguliforme, the pathology with a particular dosage is highly dependent upon the growth conditions. While the target dose is 3,300-5,000 cfu per cm of soil, the aggression of the fungus can vary widely. In this first experiment, the conditions were such that the fungus yielded an extremely aggressive infection.

Transgenic soybean lines produced as described in Examples 1 and 2 homozygous for the KP6 transgene are selected from self-crosses using quantitative PCR methods using the Promega GoTaq® qPCR master mix (Promega Corporation, Madison, Wis.) on the AB StepOne Plus-Real time PCR system (Applied Biosystems, Carlsbad, Calif.) as per the manufacturer's instructions, as described in Example 2. Their homozygous genotype is confirmed by examining expression phenotype of the progeny using U. maydis killing assays as per Gu et al. (1995) Structure 3:805-814.

For greenhouse trials, the protocol employed is that of Njiti et al. (2001) Crop Science 41:1726-1731. The Fusarium virguliforme isolate (ST90) is that isolated from sudden death syndrome (SDS)-infected roots of the soybean cultivar Spencer in Stonington, Ill., in 1990 by single spore isolation (Stephens et al. (1993) Crop Science 33:929-930). The strain, stored on Bilays medium (Bilay et al. (2000) Proc. 15th Int. Congress Sci. Cultivation of Edible Fungi, pages 757-761), is subcultured on potato dextrose agar medium (Mac Faddin (1985) Media For Isolation-Cultivation-Identification-Maintenance of Medical Bacteria, Volume 1) and used to infest a 1:1 (v/v) mixture of cornmeal and SiO. After incubation at room temperature for 10 days (O'Donnell and Gray (1995) Molecular Plant Microbe Interactions, Volume 8, pages 709-716), 5 cm³ of the inoculum is added to 250 mL of sterile water, and the average count of spores (in 10 samples of 1 mL) is determined on a hemocytometer under a microscope. Spore counts are used to calculate the volume of culture necessary for each inoculum rate. The target dosage is 3,300-5,000 cfu per cm of soil. The growth medium consists of a 1:1 (v/v) mixture of sterile sand and soil. All greenhouse experiments are planted in a randomized complete block design. Parents and non-inoculated control plants are included in the experiments. Two-week-old seedlings are transplanted onto F. virguliforme-infested plant growth medium in four-inch styrofoam cups, and kept saturated to a depth of ˜5 cm with water for 4 weeks (Njiti et al. (2001), supra).

Sudden death syndrome (SDS) is rated at 21 days after inoculation, determined on the basis of the degree of chlorosis and necrosis on each plant, and is rated (disease score, DS) on a scale of 1 to 9. A rank of 1 corresponds to 0-10% of the leaf tissue exhibiting chlorosis and 1-5% necrosis. A rank of 2 corresponds to 10-20% chlorosis and 6-10% necrosis. A rank of 3 equals 20-40% chlorosis and 10-20% necrosis. A rank of 4 equals 40-60% chlorosis and 20-40% necrosis. A rank of 5 corresponds to >60% chlorosis and >40% necrosis. A rank of 6 corresponds to up to 33% premature defoliation, and a rank of 7 represents up to 66% premature defoliation. A rank of 8 is >66% premature defoliation, and a rank of 9 represents premature death.

The results of this experiment are shown in Table 2. In this experiment, the conditions were such that there was a very aggressive infection with F. virguliforme. Indeed, in this first trial, none of the original Jack lines survive the test. The only survivors are KP6 transgenic lines. In this table, “Root IS” is the ‘root infection score’.

TABLE 2
Results of Greenhouse Trial Using a High Dose of Fusarium verguliforme
on KP6 Transgenic Soybeans

| | Week 4 | Week | | Root | RootIS | |
|---|---|---|---|---|---|---|
| Line | (0-9) | 5 | Week 6 | Wt. (g) | (0-9) | Shoot Wt. (g) |
| KP6_25-1 | 3 | 4 | 3 | 8.3 | 3 | 3.8 |
| KP6_24-1 | 1 | 2 | 1 | 28.1 | 1 | 12 |
| KP6_25-2 | 1 | 1 | 4 | 5.6 | 5 | 3.1 |
| KP6_23-1 | 3 | 2 | 3 | 4.7 | 5 | 7.4 |
| KP6_23-2 | 2 | 2 | 2 | 8.3 | 3 | 7.7 |

The lines listed in the left column denote the event number (23-25) and the last number designates the replicate number (1-2). The disease score of the foliage at various times is noted, as well as the weight and disease score of the roots. It is important to note that all of the challenged original Jack lines and the null segregants died before the first scoring at week 4.

These results demonstrate that KP6 significantly protects against SDS, and events like 24 can be remarkably resistant to high dose challenges.

These data demonstrate that line 24 exhibits very high fungal resistance, with a low disease score in both the shoot and the roots, and significant biomass in both. The other lines also showed clear patterns of resistance, in addition to surviving the challenge, but with slightly higher infection scores than line 24. These data also demonstrate that KP6 transgenic soybean lines exhibiting a high level of Fusarium resistance can be successfully selected from among transgenic events by a simple and convenient screening assay.

### Example 7

### Experiment 2

### Greenhouse Testing of KP6 Transgenic Soybean Lines Using a Medium Dose of Fusarium verguliforme on Soybean Plantlets

This experiment is performed as described in Example 6, except that a lower dose of fungus is applied to the plantlets.

While the infection used in Example 6 produces clear, qualitative results demonstrating KP6-mediated resistance to SDS, a lower dose is necessary for more quantitative analysis. As negative controls, the original non-transgenic Jack line and two null segregants (lines 31 and 32) that went through the transformation process but lost the transgene during segregation during self-crossing are used. These negative controls are compared to several lines of transgenic soybeans that are homozygous for the KP6 transgene. The results are shown in Table 3. “DS” represents ‘disease score’ as defined in Example 6. “Petiolar Ab.” refers to Petiolar Abscissions.

TABLE 3
Results of Greenhouse Trial Using a Medium Dose of Fusarium verguliforme
on KP6 Transgenic Soybeans

| | | | | | | | | |
|---|---|---|---|---|---|---|---|---|
| | DS | DS | DS | DS | Root Wt. | | | Petiolar |
| Line | wk4 | wk5 | wk6 | wk7 | (grams) | RootIS | Shoot Wt. | Ab. |

Negative Controls

| | | | | | | | | |
|---|---|---|---|---|---|---|---|---|
| Jack-1 | 2 | 2 | 3 | 2 | 13.3 | 2 | 6.4 | 0 |
| Jack-2 | 1 | 2 | 2 | 3 | 9.5 | 3 | 8.5 | 0 |
| Jack-3 | 1 | 2 | 2 | 2 | 6.1 | 3 | 6.8 | 0 |
| KP6_31-1 | 2 | 2 | 3 | 8 | 6.7 | 7 | 2.4 | 8 |
| KP6_31-2 | 1 | 2 | 2 | 9 | 2.3 | 8 | 0.9 | 6 |
| KP6_32-1 | 3 | 3.5 | 3 | 2 | 16.2 | 4 | 6.7 | 0 |
| KP6_32-2 | 2 | 3 | 3 | 2 | 18.1 | 2 | 13.8 | 0 |
| KP6_32-3 | 2 | 2 | 3 | 1 | 18.6 | 2 | 10 | 0 |

Homozygous Transgenic lines

| | | | | | | | | |
|---|---|---|---|---|---|---|---|---|
| KP6_21-1 | 1 | 3 | 3 | 1 | 10.5 | 2 | 5.5 | 0 |
| KP6_21-2 | 1 | 2 | 3 | 1 | 13.5 | 1 | 11.5 | 0 |
| KP6_21-3 | 2 | 2 | 3 | 2 | 21.3 | 1 | 12.3 | 0 |
| KP6_22-1 | 2 | 4 | 3 | 2 | 16.8 | 2 | 13.2 | 0 |
| KP6_22-3 | 1 | 2 | 3 | 2 | 10.3 | 1 | 5.4 | 0 |
| KP6_22-2 | 0 | 1 | 3 | 1 | 7.8 | 2 | 7.5 | 0 |
| KP6_23-1 | 3 | 4 | 3 | 2 | 16.7 | 1 | 9 | 0 |
| KP6_23-2 | 3 | 4 | 3 | 2 | 13.2 | 1 | 9.8 | 0 |
| KP6_23-3 | 1 | 1 | 2 | 2 | 11.7 | 2 | 5.6 | 0 |
| KP6_24-1 | 1 | 2 | 2 | 2 | 15.7 | 2 | 6.7 | 0 |
| KP6_24-2 | 1 | 2 | 2 | 2 | 21.8 | 1 | 13.5 | 0 |
| KP6_24-3 | 1 | 4 | 3 | 3 | 7.5 | 4 | 8.8 | 0 |
| KP6_25-1 | 1 | 2 | 3 | 1 | 16.7 | 1 | 6.7 | 0 |
| KP6_25-1 | 1 | 2 | 3 | 1 | 16.1 | 1 | 6.7 | 0 |
| KP6_25-2 | 2 | 2 | 3 | 2 | 14.53 | 2 | 6.6 | 0 |

While these assays are prone to significant noise, it is clear that the null (lines 31 and 32) and original Jack lines exhibit higher disease scores than the KP6 transgenic lines. In particular, KP6 transgenic lines 23 and 25 exhibit the best disease scores. These are the same lines that survived the high dose challenge in the first experiment, described in Example 6, above. It should be noted that an additional parameter, Petiolar Abscissions (“Petiolar Ab.”), is noted here. These are visible lesions that appear on the petiole due to severe infection. These are observed in null line 31.

Taken together, the data shown in Tables 2 and 3 demonstrate that KP6 offers soybean plants significant protection against SDS infection in the controlled setting of the greenhouse.

### Example 8

### Preliminary Field Trial Results

For field trials, more than 100 seed plots endemically infested by F. verguliforme were planted in Illinois locations in the spring of 2012. For each line, more than 100 seeds of nulls that were selected during segregation, and more than 100 seeds of the original Jack line of soybean, were also planted. The disease score was evaluated and analyzed as previously described in Njiti et al. (2001), supra. Triplicate plots of the homozygous KP6 transgenic and null soybean lines were planted in May, 2012 in Carbondale, Ill. Each plot was approximately 2 rows wide and approximately 6 feet long. The KP6 transgenic lines were place immediately adjacent to the various control lines. In this way, the presence of infection could be detected by the controls, and there was a transgenic event immediately adjacent for testing.

The null lines and Jack controls did not set well. Few of these plants survived the extremely dry summer 2012 growing season compared to the transgenic lines. In two different areas of the plot, a null control had clear symptoms of SDS, while immediately adjacent plots of transgenics were disease-free. The infecting pathogen was confirmed as F. verguliforme in one such case, and culturing is continuing in the other areas of the field. It is important to note that the best lines from the greenhouse trials in Examples 6 and 7 (especially line 23) showed robust growth, high yield, and freedom from disease free, and were immediately adjacent to a severely infected null line.

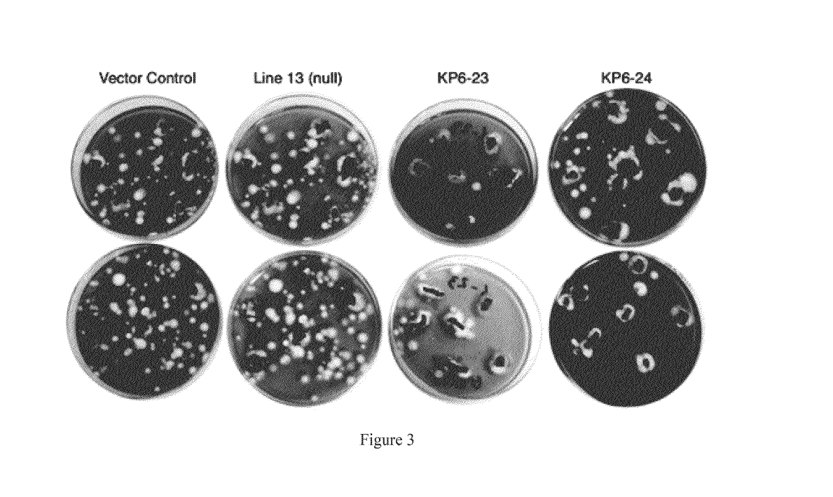

Root samples of adjacent plots were taken and assayed for F. virguliforme contamination. Portions of the roots from field-dried material were collected, rehydrated for several hours in distilled water, and placed onto agar plates containing high levels of antibiotics to kill everything but the fungus. Representative results are shown in FIG. 3. The vector control (transgenic soybeans made using the transformation vector without the KP6 transgene), and null lines (lines that went through the transformation process but were found not to have the KP6 transgene by PCR analysis) were found to have a much greater contamination level compared to the transgenic lines even though they were only inches apart in the field. In this figure, the small black pieces represent portions of the roots from the field trials. The white plaques are colonies of F. virguliforme.

As a means of summarizing all of the results from this field trial, an aggregated value of DX can be assigned to the transgenic versus non-transgenic soybeans. DX represents the overall disease score of DI*DS/100, where DI is the foliar disease incidence (number of plants with symptoms) and DS is the foliar leaf scorch disease severity (% of leaf area with symptoms). For the original Jack soybean line (control), the DX was calculated to be 20.1. The KP6-expressing lines had a DX value of 0.7. This level of protection was the same as that of other known resistant lines planted at the same time in the same field.

These data are consistent with those disclosed in Examples 6 and 7, and demonstrate that transformation of soybeans with KP6 antifungal protein results in consistent, significant fungal resistance in the same lines in all of the assays employed.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

SEQUENCE INFORMATION

Wild-Type U. maydis KP6 219 Amino Acid
Preprotoxin Sequence With Natural
N-Terminal Sequence
(SEQ ID NO: 1):
MLIFSVLMYLGLLLAGASALPNGLSPRNNAFCAGFGLSCKWECWCT
AHGTGNELRYATAAGCGDHLSKSYYDARAGHCLFSDDLRNQFYSHC
SSLNNNMSCRSLSKRTIQDSATDTVDLGAELHRDDPPPTASDIGKR
GKRPRPVMCQCVDTTNGGVRLDAVTRAACSIDSFIDGYYTEKDGFC
RAKYSWDLFTSGQFYQACLRYSHAGTNCQPDPQYE
U. maydis KP6 Preprotoxin 27 Amino Acid
Natural N-Terminal Sequence
(SEQ ID NO: 2)
MLIFSVLMYLGLLLAGASALPNGLSPR
MsDef1 Export Signal Amino Acid Sequence
(SEQ ID NO: 3)
MEKKSLAGLCFLFLVLFVAQEIVVTEA
MsDef1 Export Signal Amino Acid Sequence
Nucleotide Coding Sequence
(SEQ ID NO: 4)
ATGGAGAAGAAATCACTAGCTGGCTTATGCTTCCTCTTCTTGGTTC
TCTTTGTTGCACAAGAAATTGTGGTGACAGAAGCCA
Wild-Type U. maydis 192 Amino Acid Core KP6
Protoxin Sequence Without N-Terminal Sequence
(SEQ ID NO: 5)
NNAFCAGFGLSCKWECWCTAHGTGNELRYATAAGCGDHLSKSYYDA
RAGHCLFSDDLRNQFYSHCSSLNNNMSCRSLSKRTIQDSATDTVDL
GAELHRDDPPPTASDIGKRGKRPRPVMCQCVDTTNGGVRLDAVTRA
ACSIDSFIDGYYTEKDGFCRAKYSWDLFTSGQFYQACLRYSHAGTN
CQPDPQYE
Wild-Type U. maydis 192 Amino Acid Core KP6
Protoxin Sequence Without N-Terminal
Sequence Nucleotide Coding Sequence
(SEQ ID NO: 6)
AACAATGCTTTTTGTGCTGGATTTGGTCTCTCTTGCAAGTGGGAAT
GTTGGTGCACAGCACACGGAACGGGCAATGAATTACGGTATGCTAC
CGCAGCAGGATGCGGAGATCATCTGTCCAAGTCTTATTACGATGCT
CGGGCCGGCCACTGCCTGTTCTCTGACGACCTTCGCAACCAGTTCT
ACAGCCATTGTTCGTCTCTAAACAACAATATGTCGTGCCGGTCGTT
GTCTAAACGGACTATCCAAGATAGCGCTACCGACACGGTAGACCTC
GGTGCCGAGCTCCATAGGGATGACCCGCCCCCTACTGCTAGTGACA
TAGGCAAACGGGGTAAGAGGCCTAGACCTGTTATGTGCCAATGTGT
AGACACAACGAACGGAGGGGTTCGATTAGACGCGGTGACTAGGGCG
GCTTGCAGCATAGACTCGTTTATCGACGGGTACTATACGGAAAAGG
ATGGGTTTTGTAGAGCTAAATATTCCTGGGACTTGTTTACGAGCGG
CCAGTTCTACCAGGCATGTTTGAGGTACTCACATGCCGGGACCAAC
TGCCAACCTGACCCGCAGTATGAA
Chimeric U. maydis KP6 Protoxin Protein
Sequence With N-Terminal MsDef1 Export
Signal Amino Acid Sequence
(SEQ ID NO: 7)
MEKKSLAGLCFLFLVLFVAQEIVVTEANNAFCAGFGLSCKWECWCT
AHGTGNELRYATAAGCGDHLSKSYYDARAGHCLFSDDLRNQFYSHC
SSLNNNMSCRSLSKRTIQDSATDTVDLGAELHRDDPPPTASDIGKR
GKRPRPVMCQCVDTTNGGVRLDAVTRAACSIDSFIDGYYTEKDGFC
RAKYSWDLFTSGQFYQACLRYSHAGTNCQPDPQYE
Chimeric U. maydis KP6 Protoxin Protein
Sequence With N-Terminal MsDef1 Export
Signal Amino Acid Sequence Nucleotide
Coding Sequence
(SEQ ID NO: 8)
ATGGAGAAGAAATCACTAGCTGGCTTATGCTTCCTCTTCTTGGTTC
TCTTTGTTGCACAAGAAATTGTGGTGACAGAAGCCAACAATGCTTT
TTGTGCTGGATTTGGTCTCTCTTGCAAGTGGGAATGTTGGTGCACA
GCACACGGAACGGGCAATGAATTACGGTATGCTACCGCAGCAGGAT
GCGGAGATCATCTGTCCAAGTCTTATTACGATGCTCGGGCCGGCCA
CTGCCTGTTCTCTGACGACCTTCGCAACCAGTTCTACAGCCATTGT
TCGTCTCTAAACAACAATATGTCGTGCCGGTCGTTGTCTAAACGGA
CTATCCAAGATAGCGCTACCGACACGGTAGACCTCGGTGCCGAGCT
CCATAGGGATGACCCGCCCCCTACTGCTAGTGACATAGGCAAACGG
GGTAAGAGGCCTAGACCTGTTATGTGCCAATGTGTAGACACAACGA
ACGGAGGGGTTCGATTAGACGCGGTGACTAGGGCGGCTTGCAGCAT
AGACTCGTTTATCGACGGGTACTATACGGAAAAGGATGGGTTTTGT
AGAGCTAAATATTCCTGGGACTTGTTTACGAGCGGCCAGTTCTACC
AGGCATGTTTGAGGTACTCACATGCCGGGACCAACTGCCAACCTGA
CCCGCAGTATGAA
U. maydis KP6 α Polypeptide 79 Amino Acid
Sequence
(SEQ ID NO: 9)
NNAFCAGFGLSCKWECWCTAHGTGNELRYATAAGCGDHLSKSYYDA
RAGHCLFSDDLRNQFYSHCSSLNNNMSCRSLSK
U. maydis KP6 79 Amino Acid α Polypeptide
Nucleotide Coding Sequence
(SEQ ID NO: 10)
AACAATGCTTTTTGTGCTGGATTTGGTCTCTCTTGCAAGTGGGAAT
GTTGGTGCACAGCACACGGAACGGGCAATGAATTACGGTATGCTAC
CGCAGCAGGATGCGGAGATCATCTGTCCAAGTCTTATTACGATGCT
CGGGCCGGCCACTGCCTGTTCTCTGACGACCTTCGCAACCAGTTCT
ACAGCCATTGTTCGTCTCTAAACAACAATATGTCGTGCCGGTCGTT
GTCTAAA
U. maydis KP6 81 Amino Acid β Polypeptide
Sequence
(SEQ ID NO: 11)
GKRPRPVMCQCVDTTNGGVRLDAVTRAACSIDSFIDGYYTEKDGFC
RAKYSWDLFTSGQFYQACLRYSHAGTNCQPDPQYE
U. maydis KP6 81 Amino Acid β Polypeptide
Nucleotide Coding Sequence
(SEQ ID NO: 12)
GGTAAGAGGCCTAGACCTGTTATGTGCCAATGTGTAGACACAACGA
ACGGAGGGGTTCGATTAGACGCGGTGACTAGGGCGGCTTGCAGCAT
AGACTCGTTTATCGACGGGTACTATACGGAAAAGGATGGGTTTTGT
AGAGCTAAATATTCCTGGGACTTGTTTACGAGCGGCCAGTTCTACC
AGGCATGTTTGAGGTACTCACATGCCGGGACCAACTGCCAACCTGA
CCCGCAGTATGAATAAG

What is claimed is:

1. An antifungal composition, comprising a combination of α and β polypeptides comprising the amino acid sequences shown in SEQ ID NOs:9 and 11, respectively,
wherein said antifungal composition exhibits anti-Fusarium inhibitory activity.

2. The antifungal composition of claim 1, wherein said α and β polypeptides are present together in an antifungal effective amount.

3. The antifungal composition of claim 2, wherein said α and β polypeptides are present in stoichiometric proportion to one another.

4. The antifungal composition of claim 3, wherein said α and β polypeptides are present together in a concentration in the range of from about 0.1 microgram per milliliter to about 500 milligrams per milliliter.

5. The antifungal composition of claim 4, having a pH in the range of from about 3 to about 9.

6. The antifungal composition of claim 5, formulated with one or more additives selected from the group consisting of an inert material, a surfactant, and a solvent.

7. The antifungal composition of claim 6, formulated in a mixture of one or more other active agents selected from the group consisting of a pesticidally active substance, a fertilizer, an insecticide, an attractant, a sterilizing agent, an acaricide, a nematocide, a herbicide, and a growth regulator.

8. A method of combating, preventing, treating, controlling, reducing, or inhibiting a species of Fusarium, comprising contacting said Fusarium species with a composition comprising an antifungal effective amount of a combination of α and β polypeptides comprising the amino acid sequences shown in SEQ ID NO:9 and SEQ ID NO:11, respectively.

9. The method of claim 8, wherein said α and β polypeptides are present in said composition in stoichiometric proportion to one another.

10. The method of claim 9, wherein said α and β polypeptides are present together in said composition in a concentration in the range of from about 0.1 microgram per milliliter to about 500 milligrams per milliliter.

11. The method of claim 10, wherein said composition has a pH in the range of from about 3 to about 9.

12. The method of claim 11, wherein said composition comprises one or more additives selected from the group consisting of an inert material, a surfactant, and a solvent

13. The method of claim 12, wherein said composition further comprises a mixture of one or more other active agents selected from the group consisting of a pesticidally active substance, a fertilizer, an insecticide, an attractant, a sterilizing agent, an acaricide, a nematocide, a herbicide, and a growth regulator.

14. The method of claim 10, wherein said composition comprises microorganisms that express said α and β polypeptides.

15. A method of preventing, treating, controlling, reducing, or inhibiting Fusarium damage to a Fusarium-susceptible food crop plant, comprising expressing DNA comprising a nucleotide sequence encoding a protein comprising the amino acid sequence shown in SEQ ID NO:5 in cells thereof at a level sufficient to inhibit damage to said Fusarium-susceptible food crop plant caused by a species of Fusarium.

16. The method of claim 15, wherein said food crop plant is selected from the group consisting of maize, soybean, wheat, and sugarcane.

17. The method of claim 16, comprising:
a) inserting into the genome of a food crop plant cell a recombinant, double-stranded DNA molecule comprising, operably linked for expression:
(i) a promoter sequence that functions in plant cells to cause the transcription of an adjacent coding sequence to RNA;
(ii) a coding sequence that encodes a protein comprising the amino acid sequence shown in SEQ ID NO:5; and
(iii) a 3′ non-translated sequence that functions in plant cells to cause transcriptional termination and the addition of polyadenylation nucleotides to the 3′ end of said transcribed RNA;
b) obtaining a transformed food crop plant cell; and
c) regenerating from said transformed food crop plant cell a genetically transformed food crop plant, cells of which express said protein.

18. The method of claim 17, wherein said promoter is a root-specific promoter.

19. The method of claim 18, wherein said root-specific promoter is selected from the group consisting of RB7, RD2, ROOT1, ROOT2, ROOT3, ROOT4, ROOT5, ROOT6, ROOT7, and ROOT8.

20. The method of claim 19, wherein said Fusarium is a species selected from the group consisting of Fusarium solani, Fusarium nivale, Fusarium oxysporum, Fusarium graminearum, Fusarium culmorum, Fusarium moniliforme, Fusarium roseum, Fusarium verticillioides, and Fusarium proliferatum.
